(19) United States
(12) Patent Application Publication
(10) Pub. No.: US 2026/0207969 A1
(43) Pub. Date: Jul. 23, 2026

(54) System, Method, and Computer Readable Medium for Optimizing Radiation Treatment for a Subject

(71) Applicant: University of Virginia Patent Foundation, Charlottesville, VA (US)

(72) Inventor: Krishni Wijesooriya, Charlottesville, VA (US)

(73) Assignee: University of Virginia Patent Foundation, Charlottesville, VA (US)

(21) Appl. No.: 18/714,556

(22) PCT Filed: Dec. 1, 2022
(86) PCT No.: PCT/US2022/051592

§ 371 Date: May 29, 2024

Related U.S. Application Data
(60) Provisional application No. 63/285,150, filed on Dec. 2, 2021.

(51) Int. Cl. A61N 5/10

(52) U.S. Cl. CPC A61N 5/1031

(57) ABSTRACT

A system, method, and computer readable medium for creating an optimized Radiation Therapy (RT) treatment plan that can safely use a significantly higher dose of therapeutic radiation, and subsequently or concurrently using said plan to perform Radiation Therapy. Additionally, a method of treatment for using Radiation Therapy to safely use a significantly higher dose of therapeutic radiation. A system, method, and computer readable medium permits practitioners or users to create Radiation Therapy treatment plans that minimize the radiation doses applied to the most sensitive parts of the immune system. This technique reduces the negative immune-suppressant side-effects of treating cancer with radiation, which in turn allows practitioners to safely apply a higher dose of radiation than the prior art. Use of this technique results in treatment plans that produce significantly better outcomes for patients at no additional cost. Creating an optimized Radiation Therapy treatment plan for treating lung tumors with Stereotactic Body Radiation Therapy (SBRT) is an example of an application of the system, method, and computer readable medium.

### CROSS REFERENCE TO RELATED APPLICATIONS

The present application claims benefit of priority under 35 U.S.C § 119 (e) from U.S. Provisional Application Ser. No. 63/285,150, filed Dec. 2, 2021, entitled “Radiation Therapy Treatment Planning System and Method to Induce Tumor Reactive T Cells and to Reduce Radiation Induced Immuno Suppression to Create an In-Situ Vaccine against the Tumor”; the disclosure of which is hereby incorporated by reference herein in its entirety.

### STATEMENT OF GOVERNMENT INTEREST

This invention was made with government support under Grant no. CA234281 awarded by the National Institutes of Health. The government has certain rights in the invention.

### FIELD OF INVENTION

The present invention provides a system, method, and computer readable medium for, among other things, a radiation therapy planning technique to induce and protect T lymphocytes, B lymphocytes, Natural killer cells, and tumor antigen-specific cytotoxic immune cells such as CD8+, CD4+ cells during and following radiation therapy.

### BACKGROUND

Radiation Therapy (RT) is known to modulate the blood cells, and immune system and contribute to the generation of anti-tumor T-cells and stimulate T-cell infiltration into tumors. Some pre-clinical studies have shown that radiation facilitates dendritic cell maturation and migration (1) and an increase in tumor-reactive T-cells (2) at ablative irradiation doses. On the other hand, this anti-tumor activity may be offset by Radiation Induced Immune Suppression (RIIS) which may result in destroying this anti-tumor activity in addition to killing existing immune cells which lead to lower tumor control and survival. Lymphocytes are highly radiosensitive, and RIIS is currently considered an unavoidable side-effect of Radiation Therapy (RT). Optimizing RT treatment planning to increase the generation of tumor-reactive T-cells or protecting them from radiation by reducing RIIS has been never studied. If the present inventor can balance the creation of anti-tumor immunity, new cytotoxic T-cells (in particular CD4+ and CD8+ T-cells which are specific to a particular patient's neo-antigens) while managing to reduce the RIIS the present inventor may be able to retain this tumor specific adaptive immunity to fight against the tumor as well as distant metastasis, creating an in-situ vaccine against the tumor. All this has to occur while delivering a tumorcidal RT dose to the Planning Target Volume (PTV).

Specifically, this technique will ensure or increase the probability of observation of predicted T-cell priming effect of SBRT, abscopal effect, increase of CD8+ and CD4+ T-cell dependent immunity that led to tumor eradication and relapse reduction. Immunotherapies are only to break the inhibitory mechanisms using Immune Checkpoint Blockade (ICB) therapies using anti-bodies such as anti-PD1, anti-PD_L1, and anti-CTLA4. Immunotherapy relies on the presence of neo-antigen specific cytotoxic T-cells in the body. Therefore, this planning technique by stimulating T-cell production in the body will create an immune hot environment increasing the efficacy of immunotherapy. With the increasing concurrent use of immunotherapy and radiation for cancer patients, radiation treatments that uses this planning technique to create new tumor antigen-specific cytotoxic immune cells will be able to irradicate the tumor and create a tumor vaccine with or without the additional help of immunotherapy to overcome inhibitory checkpoints. Additionally, this treatment planning technique will reduce immune suppression due to radiation that can lead to less hospitalizations and medications leading to less health care costs and better survival.

Ionizing radiation is generally used as a powerful tool to specifically kill cancer cells at target sites by DNA damage. It is also known to induce immunosuppression and lymphopenia which may result in lower tumor control and survival. Lymphopenia caused by radiation therapy was first described in the early 20th century, just shortly after the discovery of x-rays (7). It has been demonstrated that radiation can induce lymphopenia in the absence of concomitant chemotherapy or steroids, and even when neither bone marrow nor lymphatic tissue is included in the treatment field. Studies have shown that irradiation of the brain, which includes minimal bone marrow in the calvarium and no lymphatic tissue, can cause a greater than 60% decrease in lymphocyte count (8). One study demonstrated that irradiation of circulating blood with cesium placed inside a shielded dialysis unit caused a 60% to 80% drop in the number of circulating lymphocytes that persisted for many years after radiation exposure (9). Therefore, it is well established that irradiation of circulating blood reduces lymphocyte counts significantly.

Recent studies have shown a correlation between Treatment Related Lymphopenia (TRL) and inferior survival in patients with glioblastoma, advanced stage non-small cell lung cancer (NSCLC), pancreatic cancer and squamous cell carcinoma of the head and neck (10)(11)(12)(13)(14). In a study published in 2015 (15), investigators collected and analyzed data from 4 independent solid tumor sites from each of 297 patients with newly diagnosed malignant glioma, resected and un-resected pancreatic cancer, and stage III NSCLC. The investigators recorded lymphocyte counts, prognostic factors, treatment and survival. They defined TRL as <500 cells/mm3 and found an increased risk for death attributed to TRL in each cohort. They observed severe TRL in 40% of patients 2 months after the initiation of chemoradiation and found that it was independently associated with shorter survival from tumor progression (see FIG. 1).

In a study looking at patients treated for squamous cell carcinoma of the head and neck, it was shown that at two months 60% of patients had severe TRL which was independently associated with earlier disease progression than those with lower TRLs (HR 5.75, p=0.045) (14). In another study that examined the effect of steroids and RT on the lymphocyte count in patients treated for primary brain tumors, it was found that 17 of the 70 (24%) patients had CD4 counts that decreased to <200/mm3 and that these patients were more likely to be hospitalized (41% vs 9%, p<0.01) with 23% vs 4%, p<0.05 hospitalized for infection (16). In another study, Kaplan-Meir curves (17) showing survival for 133 patients that underwent treatment for locally advanced pancreatic cancer were stratified by severe lymphopenia (Total Lymphocyte Count (TLC)<500 cells/mm3) two months after starting radiation therapy. They reported a statistically significant survival difference for patients with higher TLC (see FIG. 2). Median survival for patients with severe lymphopenia at 2 months after starting RT was 12.4 months (95% CI: 8.7-16.1) versus 15.2 months (95% CI: 12.7-17.9) for patients with TLC>500 cells/mm3 (P=0.055) (18).

Example I: i) Early-stage lung cancer patients who are ineligible for surgery currently undergo Stereotactic Body Radiation Therapy (SBRT) typically with a dose fractionation of 60Gy in 5 fractions or 50Gy in 5 fractions. Comparison of 30 retrospective single arm studies, including 2611 patients, between photon/proton, carbon-ion treatments for stage I NSCLC show that 2 and 5 years overall and disease specific survival following SBRT is around 70%: 42% (overall), and 83%: 63% (disease specific) (3). ii) Our analysis with retrospective data shows a hazard ratio of 2.11 (95% CI 1.26-3.54, P=0.005) for patients with post-treatment absolute lymphocyte counts<0.5×10⁹ cells/L compared to the group with absolute lymphocyte counts (ALC)≥0.5×10⁹ cells/L (4). iii) Our preliminary data from the clinical trial show a 46%, 20%, and 26% increase of post-treatment Absolute Lymphocyte Counts (ALC) at 5 days, 4 weeks, and 6 months with the optimized group described above compared to the standard arm that simply meet RTOG0915/0813 criteria (5). Therefore, this dosimetric workflow will lead to higher overall survival for the optimized arm patients.

Example II: Although local control is high for patients with operable NSCLC, systemic distant relapse remains the predominant failure pattern. Even among patients with the earliest clinical stage of lung cancer, 50% will die within 5 years of diagnosis after lobectomy (6). For patients with Stage II and IIIA disease and good performance status, platinum-based chemotherapy is recommended to improve systemic relapse rates. For the subset of early-stage patients with identifiable poor prognostic characteristics treated with surgery, immunotherapy can be integrated with RT planned according to this invention (carefully planned to reduce RIIS and increase neo-antigen specific cytotoxic T-cells) at the optimum time point post RT to improve systemic relapse by creating memory T-cells that are specific to that patient's neo-antigens.

There is therefore a need in the art for an effective technique for improving radiation therapy treatment plan for a subject and to provide better modes of treatment.

### SUMMARY OF ASPECTS OF EMBODIMENTS OF THE PRESENT INVENTION

An aspect of an embodiment of the present invention relates to, but not limited thereto, a radiation therapy treatment planning technique using any type of radiation therapy delivery (photons, protons/heavy ions), machine/planning system combination, for patient specific, and tumor location specific tumor reactive T-cell creation and reduction of any type of blood cell kill related to radiation therapy treatments. As an example, lymphocyte kill following Stereotactic Body radiation therapy (SBRT) to lung tumors is described here. This novel technique optimizes the increase of creating anti-tumor immunity: new cytotoxic T-cells (in particular CD4+ and CD8+ T-cells which are specific to a particular patient's neo-antigens) while managing to reduce the radiation induced immune suppression (RIIS) especially from the circulating blood, bone marrow, lymph nodes (especially the tumor draining lymph node where the highest probability of the dendritic cells meet with the naïve T-cells, antigen presentation and finally the clonal expansion occur).

An aspect of an embodiment of the present invention provides a system, method, and computer readable medium for, among other things a radiation therapy treatment planning technique to induce tumor reactive T-cells and to reduce radiation induced immune suppression (RIIS) to create an in-situ vaccine against the tumor.

An aspect of an embodiment of the present invention provides, among other things, a computer method for determining a radiation therapy (RT) treatment plan for a subject. The method may comprise:
a) determining, using subject data for the subject and a radiation therapy (RT) treatment plan simulation model, an internal target volume (ITV) mean fraction dosage within a first specified amount of radiation dose for a specified number of radiation therapy (RT) fractions;
b) determining, using subject data and the RT treatment plan simulation model, a total dose and/or an equivalent fractional dose to the heart that is less than a second specified amount of radiation;
c) determining, using the subject data and the RT treatment plan simulation model, a total dose and/or an equivalent fractional dose to one or more specified blood-rich organs that is less than a third specified amount of radiation;
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation;
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘f’; and
h) outputting said RT treatment plan for use in concurrently or subsequently treating the subject.

An aspect of an embodiment of the present invention provides, among other things, a system for use in determining a radiation therapy (RT) treatment plan for a subject. The system may comprise: a computer processor; and a memory configured to store instructions that are executable by said computer processor. Further, wherein said computer processor is configured to execute the instructions for:
a) determining, using subject data for the subject and a radiation therapy (RT) treatment plan simulation model, an internal target volume (ITV) mean fraction dosage within a specified amount of radiation dose for a first specified number of radiation therapy RT fractions;
b) determining, using the subject data and the RT treatment plan simulation model, a total dose and/or an equivalent fractional dose to the heart that is less than a second specified amount of radiation
c) determining, using the subject data and the RT treatment plan simulation model, a total dose and/or an equivalent fractional dose to one or more specified blood-rich organs that is less than a third specified amount of radiation
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘; and
h) outputting said RT treatment plan for use in concurrently or subsequently treating the subject.

An aspect of an embodiment of the present invention provides, among other things, a non-transitory, computer readable storage medium having instructions stored thereon for use in determining a radiation therapy (RT) treatment plan for a subject, that, when executed by a computer processor, cause the computer processor to:
a) determine, using subject data for the subject and a radiation therapy (RT) treatment plan simulation model, an internal target volume (ITV) mean fraction dosage within a specified amount of radiation dose for a first specified number of radiation therapy (RT) fractions;
b) determine, using the subject data and the RT treatment plan simulation model, a total dose and/or an equivalent fractional dose to the heart that is less than a second specified amount of radiation
c) determine, using the subject data and the RT treatment plan simulation model, a total dose and/or an equivalent fractional dose to one or more specified blood-rich organs that is less than a third specified amount of radiation
d) determine, using the subject data and the RT treatment plan simulation model, a total dose and/or an equivalent fractional dose to one or more specified bone-marrow-rich organs that is less than a fourth specified amount of radiation
e) determine, using the subject data and the RT treatment plan simulation model, a total dose and/or an equivalent fractional dose to one or more specified secondary lymphoid organs that is less than a fifth specified amount of radiation
f) determine, using the subject data and the RT treatment plan simulation model, a total dose and/or an equivalent fractional dose to a tumor draining lymph node that is less than a sixth specified amount of radiation;
g) determine the radiation therapy (RT) treatment plan for the subject using results from the processing steps ‘a’ through ‘f’; and
h) output said RT treatment plan for use in concurrently or subsequently treating the subject.

An aspect of an embodiment of the present invention provides, among other things, a method for treating a subject with an optimizing radiation therapy (RT) treatment, whereby the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
whereby the subject is treated with an optimized RT treatment.

An aspect of an embodiment of the present invention provides, among other things, a method for reducing the risk of developing Treatment Related Lymphopenia (TRL) and/or radiation induced immune suppression (RIIS) in a subject undergoing a cancer and/or a tumor treatment. The method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment reduces the risk of developing TRL and/or RIIS in the subject relative to a standard RT treatment.

An aspect of an embodiment of the present invention provides, among other things, a method for inducing tumor reactive T cells in a subject undergoing an anti-tumor treatment. The method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment induces tumor reactive T cells in the subject.

An aspect of an embodiment of the present invention provides, among other things, use of an optimized radiation therapy (RT) treatment for treating a subject with a disease, disorder, or condition for which radiation therapy would be appropriate, and/or for reducing the risk of developing Treatment Related Lymphopenia (TRL) and/or radiation induced immune suppression (RIIS), and/or for inducing reactive T cells. Further, wherein the optimized radiation therapy (RT) treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
and further wherein the optimized radiation therapy (RT) treatment treats the disease, disorder, or condition; reduces the risk of developing Treatment Related Lymphopenia (TRL) and/or radiation induced immune suppression (RIIS); and/or induces reactive T cells in the subject.

An aspect of an embodiment of the present invention provides, among other things, a system, method, and computer readable medium for creating an optimized Radiation Therapy (RT) treatment plan that can safely use a significantly higher dose of therapeutic radiation, and subsequently or concurrently using said plan to perform Radiation Therapy. Additionally, a method of treatment for using Radiation Therapy to safely use a significantly higher dose of therapeutic radiation. A system, method, and computer readable medium permits practitioners or users to create Radiation Therapy treatment plans that minimize the radiation doses applied to the most sensitive parts of the immune system. This technique reduces the negative immune-suppressant side-effects of treating cancer with radiation, which in turn allows practitioners to safely apply a higher dose of radiation than the prior art. Use of this technique results in treatment plans that produce significantly better outcomes for patients at no additional cost. Creating an optimized Radiation Therapy treatment plan for treating lung tumors with Stereotactic Body Radiation Therapy (SBRT) is an example of an application of the system, method, and computer readable medium.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features, and advantages of the present invention, as well as the invention itself, will be more fully understood from the following description of preferred embodiments, when read together with the accompanying drawings

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 is a graphical representation demonstrating the relationship between survival and grade III/IV TRL and the association between severe TRL in 40% of patients two months after the initiation of chemoradiation with shorter survival from tumor progression.

FIG. 2 is a graphical representation demonstrating a statistically significant survival difference for patients with higher TLC.

FIG. 3 is a schematic illustration of the cancer immunity cycle with stimulatory factors and inhibitory factors.

FIG. 4 is a schematic illustration of radiation induced modulation (48).

FIG. 5(A), top panel, is a simulated diagram of the superior view [towards the head] from a transverse or axial [horizontal plane dividing top and bottom] cross-section of a subject, with simulated organs showing the inclusion of all organs in the thorax showing the dose distribution for two centrally located tumors; and FIG. 5(A), bottom panel, is graphical representation of the dose volume histogram (DVH).

FIG. 5(B), top panel, is a simulated diagram of the superior view [towards the head] from a transverse or axial [horizontal plane dividing top and bottom] cross-section of a subject, with simulated organs showing the inclusion of all organs in the thorax showing the dose distribution for two centrally located tumors; and FIG. 5(B), bottom panel, is graphical representation of the dose volume histogram (DVH).

FIG. 6(A), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(A), bottom panel, is graphical representation of the dose volume histogram (DVH).

FIG. 6(B), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(B), bottom panel, is graphical representation of the dose volume histogram (DVH).

FIG. 6(C), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(C), bottom panel, is graphical representation of the dose volume histogram (DVH).

FIG. 7 is a graphical representation of the preliminary results showing the reduction of immune suppression in the optimized arm at all three time points.

FIG. 8 is a graphical representation of the percentage increase of CD4+ & CD8+ T-cells from baseline as a function of mean dose to tumor.

FIG. 9 is a simulated diagram of the superior view [towards the head] from a transverse or axial [horizontal plane dividing top and bottom] cross-section of a subject, with simulated organs showing the inclusion of all organs in the thorax showing the dose distribution for two peripherally located tumors.

FIG. 10 is a block diagram illustrating an example of a machine upon which one or more aspects of embodiments of the present invention can be implemented.

FIGS. 11(A)-(B) is a flow diagram of a method for providing dosimetric criteria for optimizing a radiation therapy (RT) treatment plan for a subject.

FIGS. 12(A)-(B) is a flow diagram of a method for treating a subject by providing dosimetric criteria for optimizing a radiation therapy (RT) treatment on said subject.

FIG. 13 schematically illustrates a radiation therapy system treating a subject and a molecular imaging system to image or scan the subject.

FIG. 14 schematically illustrates an immunotherapy system, a surgical apparatus, system, instrument or device, or a chemotherapy system in communication with the subject.

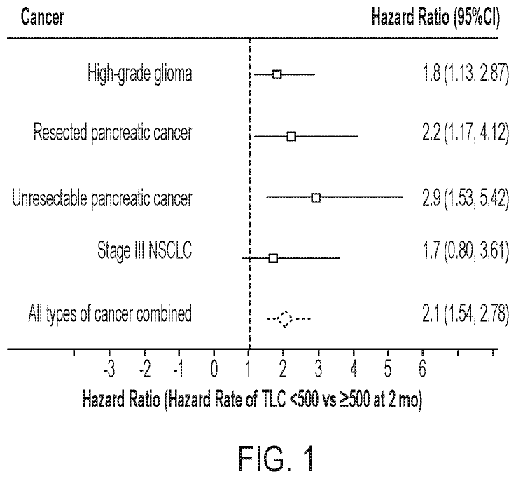

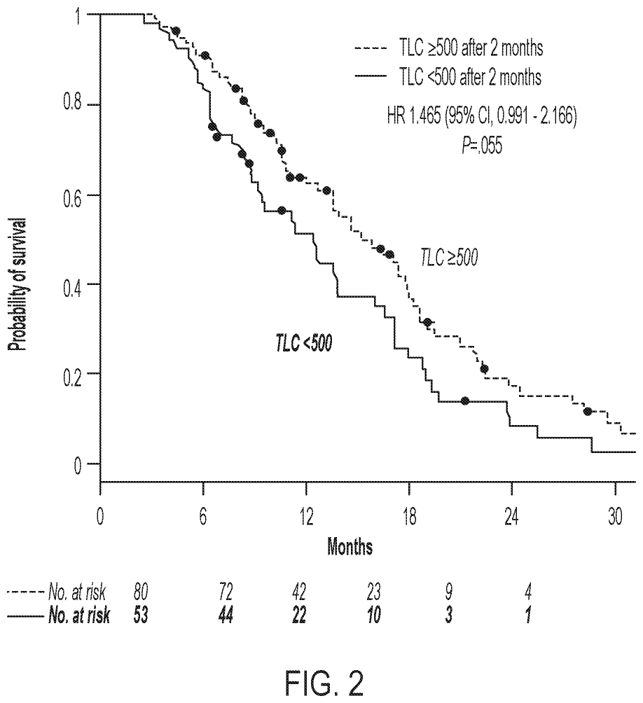

### DETAILED DESCRIPTION OF ASPECTS OF EMBODIMENTS OF THE PRESENT INVENTION

### Tumor Microenvironment:

The microenvironment of the tumor consists of innate immune cells which include: Natural Killer (NK) cells, neutrophils, macrophages, mast cells, myeloid-derived suppressor cells (MDSCs), and Dendritic Cells (DC) as well as adaptive immune cells which are the T and B lymphocytes (19). It has been suggested that infiltration of the primary tumor by memory T-cells, particularly of the Th1 and cytotoxic types, is the best prognostic factor of disease-free survival and overall survival at all stages of clinical disease (20).

### Cancer Immunity Cycle:

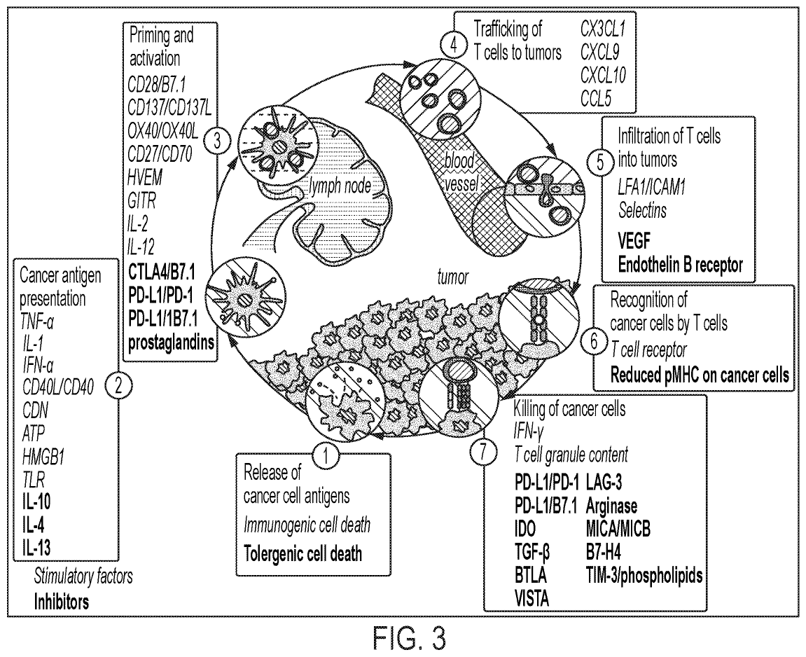

Immune cells are constantly surveilling our body and eliminating tumors as given in FIG. 3. FIG. 3 provides a schematic of the cancer immunity cycle with stimulatory factors and inhibitory factors given in italics and non-italics, respectively (21). But in some other occasions, immune cells are induced to become exhausted, and the tumors will grow. Tumor antigens are the targets of the immune defense against cancer. The immune system uses antibodies and T-cells to recognize and kill cancer cells. Antibodies called plasma cells are made by B-cells and they will recognize any chemical form of antigen that a tumor can make as long as they are on the surface of the cancer. They cannot get inside and see antigens inside the cancer. CD8+ T-cells will recognize peptide fragments or proteins that are made inside a tumor cell and placed on a Human Leukocyte Antigen (HLA) molecule. CD8+ cytotoxic T-cells kill cancer cells by the direct release of cytotoxic substances that they store in granules called perforins, and granzymes. CD4+ helper T-cells will recognize peptide fragments of proteins that are taken up from the cancer cell by another cell such as an antigen presenting cell (macrophage). This helps the macrophages kill the tumor cells, and will lead to apoptotic death of the cancer cell.

### Immunotherapy in the Fight Against Cancer:

Immune checkpoint proteins, such as the inhibitory receptor called Cytotoxic T Lymphocyte (CTL) A4, can inhibit the development of an active immune response by acting primarily at the level of T-cell development and clonal expansion at the lymph node (level 3 in the cancer immunity cycle). The activated T-cells in the lymph node can induce on their surface a molecule called CTLA4. Due to CTLA4, there will be an attenuation of the clonal expansion process and the immune system will be dampened. On the other hand, proteins such as PD-L1 and PD-1 can have an inhibitory function that primarily acts to modulate active immune responses in the tumor bed. PD-L1 in the tumor binds to PD-1 on the effector T-cell and make the T-cell exhausted. Similarly, Tregs will exhaust the effector T-cells at the tumor site by expressing large amounts of CTLA4. By giving Immune Checkpoint Blockade (ICB) therapy like anti-CTLA-4, anti-PD-L1, or anti-PD1 antibodies, one could counteract each one of the four above mentioned immune system dampers to some extent (22). Therefore, ICB therapy can help eradicate the tumor only if the effector T-cells are somehow created.

### Vast Heterogeneity of Signals Sent by the Cancer Cells to the Immune System:

Every one of the tumors on different people, even on the same organ, will be immunogenically different from each other. There may be some common driver mutations that are frequently associated with cancer development. In the case of lung cancer these may be KRAS or epidermal growth factor receptor. But the passenger mutations are generally random and have nothing to do with the phenotype, which will be different for each patient. Furthermore, each patient will have different MHC (Major Histocompatibility Complex) or HLA molecules because HLA molecules are highly polymorphic and different unless they are from twins. Since the peptides bind to HLA alleos, the different combination of neo-antigens and the different HLA alleols will lead to every different lung cancer in each of these patients being different in terms of what it is showing to the immune system. So this high degree of heterogeneity among cancers in terms of what they show to the immune system is the reason why individualized vaccines are needed. The T-cell response to cancer is mainly for the passenger mutations simply because there are many more of them than the driver mutation. But T-cell could certainly respond to the neo-antigens that are part of driver mutation.

### Role Played by Lymphocyte Subsets in Anti-Tumor Immunity:

In addition to their vital function in the body's general defenses against infections, lymphocyte sub types also play very important roles in tumor suppression. It has been shown that the expressions of CD3+ and CD4+ sub types of lymphocytes were significantly associated with overall survival of NSCLC patients (23). CD8+ and CD56+ cells exert antitumor activity via antigen specific and antigen nonspecific mechanisms (24) (25). Elevated circulating CD19+ lymphocytes can predict survival in patients with gastric cancer (26). There have been many other studies which have shown that CD3+, CD4+, CD8+, CD19+, and CD56+ subsets are important in antitumor immunity, and immune suppression may increase the risk of tumor growth and metastasis (27)(28)(29)(30)(31)(32). Therefore, the reduction of RT induced suppression of these lymphocyte subsets has the potential for decreasing tumor growth and metastasis.

### Evidence of Radiation Creating an Anti-Tumor Immune Response:

Beyond the direct effects of radiation, localized radiation can have distant bystander effects that causes tumor suppression outside of the irradiated region (33). This abscopal effect would be a very beneficial phenomenon, either to treat pre-existing distant metastases or to treat residual disease that was not removed by the primary treatment regimen. Furthermore, many authors have reported that RT leads to significant positive changes in the tumor microenvironment, especially with respect to tumor infiltrating immune cells. Post radiation therapy (RT), some patients and animal subjects have been shown to develop tumor antigen-specific immune responses that were not observed prior to treatment; this seems to suggest that in some patients there is a strong immune increase following RT while in some other patients an immune decrease is observed (34). This phenomenon randomly observed by multiple authors at multiple tumor sites is not relative, and could be enabled to occur in every patient and every tumor site by carefully crafted dose distributions during RT treatment planning.

### Using Radiation Therapy to Create an In-Situ Vaccine:

Much of the promising work in tumor immunotherapy has been focused on T-cell-mediated, antigen-specific vaccines. Previous research has shown that through cellular immunotherapy, T-cells can destroy large, established tumors. If the present inventor can balance the creation of anti-tumor immunity, new cytotoxic T-cells (in particular CD4+ and CD8+ T-cells which are specific to a particular patient's neo-antigens) while managing to reduce the radiation induced immune suppression (RIIS) especially from the circulating blood, bone marrow, lymph nodes (especially the tumor draining lymph node where the highest probability of the dendritic cells meet with the naïve T-cells, antigen presentation and finally the clonal expansion occur) the present inventor may be able to retain this tumor specific adaptive T-cell immunity to fight against the tumor as well as distant metastasis, creating an in-situ vaccine against the tumor.

### Role of Radiation Therapy in the Process of Creating New Cytotoxic T-Cells:

The abscopal effect is not typically observed with RT alone (35). Evidence supporting the role of RT in promoting cross-priming and the induction of anti-tumor T-cell responses was suggested earlier on by an experimental mouse model (36). Effective initiation of the adaptive immune system requires antigen presentation by professional Antigen Presenting Cells (APC)s. DC maturation is critical for APCs to effectively present antigens to naïve T-cells in draining lymph nodes. However, apoptotic cells efficiently load DCs with the tumor antigen, but do not cause DC maturation (37). By contrast, antigen from non-apoptotic cells also loads DCs, but also causes DCs to mature and upregulate costimulatory molecules (38). The current understanding is that radiation could kill tumor cells, releasing tumor antigens and molecules with what are collectively called Damage-Associated Molecular Patterns (DAMPs). DAMPs exert various immunomodulatory effects, including induction of the expression of cytokines and chemokines, and release of inflammatory mediators (39). Radiation also increases the permeability of the local vasculature either directly or through cytokine production. This leads to the recruitment of circulating leukocytes into surrounding tissues including APCs and effector T-cells (40).

As with many other challenges, as shown in ref (22), radiation upregulates expression of immunomodulatory surface molecules (MHC, costimulatory molecules, adhesion molecules, death receptors, heat shock proteins) and secretory molecules (cytokines, inflammatory mediators) in tumor, stromal, and vascular endothelial cells. With a murine colon adenocarcinoma model, Reits et al. (41) data support that radiation enhances MHC class I expression which is a necessary step in antigen presentation as well as recognition of the tumor by the cytotoxic T-cells (CTLs).

Toll-like receptor 4 (TLR4) expression by DCs also appears to be a prerequisite for efficient antigen presentation of tumor antigens furnished by dying cancer cells (42). Apetoh et al. data show that radiation can trigger signals that stimulate TLR4 on antigen-presenting DCs (42). Liao et al. (43) have shown the enhancement of presentation of antigenic peptides when DCS are irradiated and it is a maturation signal, while inhibiting internal antigen processing. Merrick et al. (44) have shown a decrease in IL-12 production that has a negative effect on presentation. Several reports have shown increased expression of MHC class I and co-accessory molecules after radiation of both tumor and host cells, while Chakraborty et al. (45) reported a direct effect of radiation on tumors by modifying the phenotype of tumor cells to render them more susceptible to vaccine-mediated T-cell killing. Others have shown that radiation-induced changes in the tumor immune microenvironment promotes greater infiltration of immune effector cells (46). This is excellent news since in our body only a couple of T-cell receptors have the ability to recognize a given neo antigen. Having the tumor cells produce many different mutated neo antigen types that are different from self antigens, i.e. different phenotypes with radiation will increase the probability of cloning and priming T-cells with multiple receptor types leading to better manage or irradicate the tumor and to create an in-situ vaccine. The peptides that are generated from mutated proteins that are put on HLA molecules will be seen by T-cells because we are not tolerant to these neo-antigens. That will activate the T-cell and will create many more T-cells made specific for that peptide, which will attack the tumor.

### Radiation Therapy Treatment Planning Dosimetry on Immune Cells:

Although lymphocyte radiosensitivity is well recognized, the knowledge of how different doses and delivery methods impact systemic and locoregional naive, effector, or Treg or other immunologically relevant populations is still in its infancy. Available literature on the potential immunomodulatory effects of localized RT on tumors are conflicting as to whether these responses promote or interfere with tumor reduction. This dualism is something that is to be expected until we learn to optimize radiation therapy planning that lead to tumor reduction and improve survival.

Mice bearing a syngeneic mammary carcinoma, 67NR, in both flanks were treated with Flt3-L after local irradiation with a single dose of 2-6Gy to only one of the two tumors (36). The growth of the non-irradiated tumor was also impaired by the combination of RT and Flt3-L. Reitz et al. (41) showed that anti-tumor immunotherapy with adoptive CTL cells is active only when preceded by RT (8-10Gy) of the primary tumor. Shiraishi et al. (47) showed the marked infiltration of CD4+ and CD8+ cells, not only in the irradiated site, but also at the non-irradiated site and the presence of with ECI301 (a chemokine secreted by various leukocytes, including T lymphocytes and activated macrophages and recruiting certain cells such as monocytes and DCs) after local irradiation of 6Gy. Lee et al. demonstrated that CD8 T-cell responses play an important role in the therapeutic outcome of RT in animal models (1). Lee et al. (1) observed that local RT on grafted tumors generates CD8+ T-cell immunity to lead to tumor reduction, local relapse reduction, and even eradication of metastasis in some settings. An increase of infiltrating T-cells in the tumor microenvironment and the draining lymphoid tissues was seen 1-2 weeks after treatment with higher radiation dose (15-20Gy in one to four fractions). The delivery of an ablative dose of radiation of 15-25Gy was found to cause a significant increase in T-cell priming in draining lymphoid tissue, leading to reduction or eradication of the primary tumor or distant metastasis in a CD 8+ fashion in an animal model.

Looking at these results from the prior research it is unclear what the optimum dose per fraction to create cytotoxic T-cells could be, and it could be anywhere between 2Gy-20Gy.

Aspects of embodiments of the present disclosure relate to, among other things, methods and systems of reducing Radiation Induced Immune Suppression (RIIS) and generating tumor reactive T-cells.

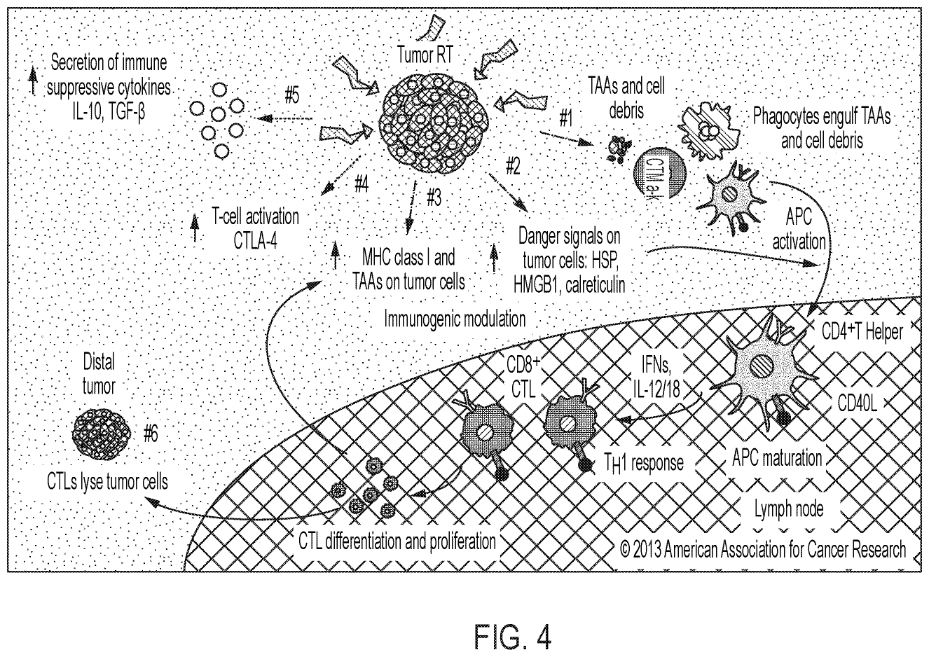

FIG. 4 is a schematic illustration of radiation induced modulation (48). Lymphocytes are a key component of the adaptive immune system. Radiation may overcome mechanisms of tumor immune escape. Radiation therapy (RT) is known to modulate the immune system, contribute to the generation of anti-tumor T-cells and stimulate T-cell infiltration into tumors. Ionizing radiation can induce anti-tumor immunity via causing the secretion of various danger-associated molecular pattern (DAMP) molecules, which can stimulate dendritic cells and cytotoxic T-cells. Some pre-clinical studies have shown that radiation facilitates dendritic cell maturation and migration (1) and an increase in tumor-reactive T-cells (2) at ablative irradiation doses. On the other hand, this anti-tumor activity may be offset by Radiation Induced Immune Suppression (RIIS) which may result in lower tumor control and survival. Lymphocytes are highly radiosensitive, and RIIS is currently considered an unavoidable side-effect of RT. Current national or international radiation therapy protocols are oblivious to RIIS or generating the anti-tumor T-cells. Doses to different organs affect the immune suppression in different ways. The maximum dose and the mean dose for a radiation plan are not necessarily the best parameters for evaluating the level of RIIS. Instead, the dynamics between the time dependent dose to structures and velocities of blood through those organs need to be carefully taken into account to minimize the expected immune cell loss for a given RT plan. Furthermore, one needs to have a good understanding of the process, dosimetry, anatomical locations, timing of the naïve T-cells turning in to effector T-cells via clonal expansion, and how long it takes for these newly created T-cells to exit the lymph node. Optimizing RT treatment planning either to increase the generation of tumor-reactive T-cells or to reduce RIIS had been never studied. If the present inventor can balance the creation of anti-tumor immunity and new cytotoxic T-cells (in particular CD4+ and CD8+ T-cells which are specific to a particular patient's neo-antigens), while managing to reduce the RIIS, especially from the circulating blood, bone marrow, lymph nodes the present inventor can retain this tumor specific adaptive T-cell immunity to fight against the tumor as well as distant metastasis, creating an in-situ vaccine against the tumor. In addition to the lymph nodes where the highest population of CD8+ naïve T-cells reside (22), the tumor draining lymph node is where it is most likely for the dendritic cells to meet with the naïve T-cells, and for antigen presentation and finally the clonal expansion occur. It has been shown that tumor-draining lymph nodes are a rich source of tumor antigen-specific T-cells (49). Therefore, it is crucial to save the lymph node from radiation. All this has to occur while delivering a tumorcidal RT dose to the PTV.

The present inventor has managed, for the first time, to achieve this in a prospective clinical trial of 16 lung SBRT patients. There were two arms of the trial: the treatment plans in the standard arm were created following dosimetric criteria from RTOG 0915 and 0813, while the treatment plans in the optimized arm were further optimized to minimize immune suppression and increase the production of cytotoxic T lymphocytes. This analysis showed i) that not every SBRT radiation therapy treatment plan is equivalent just because they meet the national protocol standards in modulating the immune system for the benefit of the patient, ii) that one could generate the anti-tumor T-cells following SBRT to lung by carefully selecting the dose distributions to minimize the dose to tumor draining lymph node where these clonal expansions occur while giving a five fraction dose greater than 70Gy to the tumor, and iii) that one could reduce the RIIS by reducing dose to circulating lymphocytes and not disturbing the bone marrow hematopoiesis and secondary lymphoid organs. The resulting elevated total and sub-type lymphocyte counts could be preserved even at longer time intervals post SBRT to lung (Wijesooriya technique, i.e., technique of the present inventor).

In some cases, for example but not limited thereto, the present inventor used the following set of criteria to develop a set of RT treatment planning guidelines that lead to creation of tumor reactive T-cells, while minimizing RIIS planning for thorax RT:
1. Keep the ITV mean dose at 14-17Gy per fraction.
2. Keep the total dose to heart to less than 5Gy.
3. Keep the total dose to the aorta, vena cava, pulmonary artery to less than 8Gy
4. Keep the total dose to thoracic spine less than 2Gy.
5. Keep the total dose to lymph nodes/lymphatics less than 2Gy.
6. Keep the total dose to tumor draining lymph node to less than 2Gy.

In an embodiment, an aspect of an embodiment provides, but not limited thereto, a method or system for providing dosimetric criteria for creating an optimized radiation therapy (RT) treatment plan for a subject.

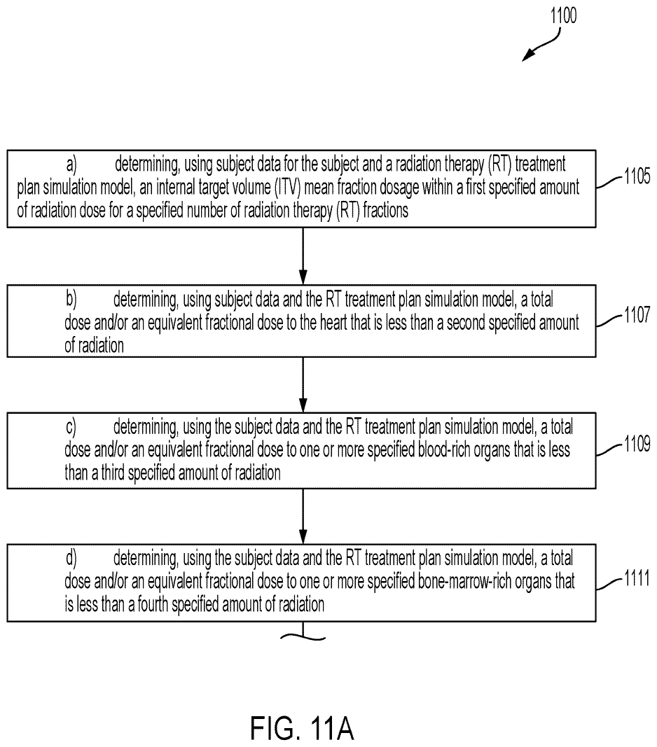

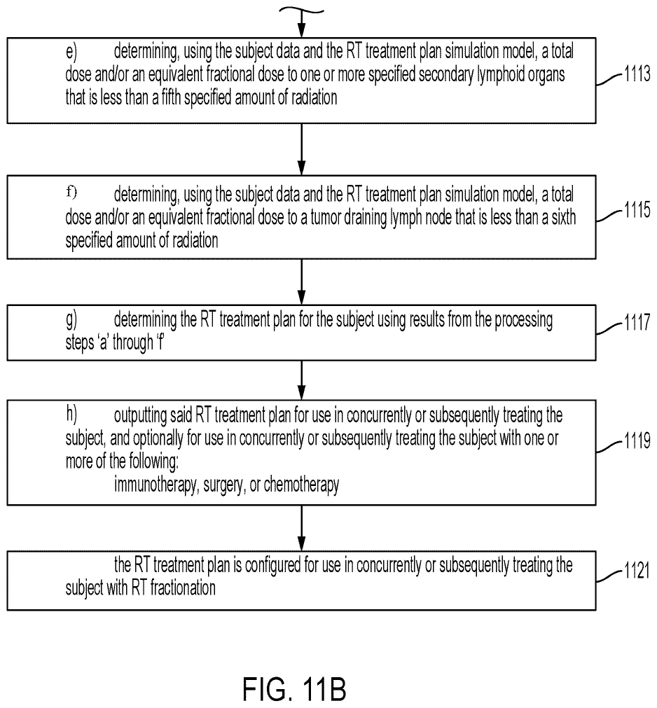

FIGS. 11(A)-(B) is a flow diagram of a method 1100 for determining a radiation therapy (RT) treatment plan for a subject. The method 1100 can be performed by a system of one or more appropriately-programmed computers in one or more locations. At step 1105, the method includes a) determining, using subject data for the subject and a radiation therapy (RT) treatment plan simulation model, an internal target volume (ITV) mean fraction dosage within a first specified amount of radiation dose for a specified number of radiation therapy (RT) fractions. At step 1107, the method includes b) determining, using subject data and the RT treatment plan simulation model, a total dose and/or an equivalent fractional dose to the heart that is less than a second specified amount of radiation. At step 1109, the method includes c) determining, using the subject data and the RT treatment plan simulation model, a total dose and/or an equivalent fractional dose to one or more specified blood-rich organs that is less than a third specified amount of radiation. At step 1111, the method includes d) determining, using the subject data and the RT treatment plan simulation model, a total dose and/or an equivalent fractional dose to one or more specified bone-marrow-rich organs that is less than a fourth specified amount of radiation. At step 1113, the method includes e) determining, using the subject data and the RT treatment plan simulation model, a total dose and/or an equivalent fractional dose to one or more specified secondary lymphoid organs that is less than a fifth specified amount of radiation. At step 1115, the method includes f) determining, using the subject data and the RT treatment plan simulation model, a total dose and/or an equivalent fractional dose to a tumor draining lymph node that is less than a sixth specified amount of radiation. At step 1117, the method includes g) determining the RT treatment plan for the subject using results from the processing steps ‘a’ through ‘f’. At step 1119, the method includes h) outputting said RT treatment plan for use in concurrently or subsequently treating the subject. Moreover, still referring to step 1119, in an embodiment the method may optionally include for use in concurrently or subsequently treating the subject with one or more of the following: immunotherapy, surgery, or chemotherapy. Moreover, at step 1121, the method includes providing a using the RT treatment plan which is configured for use in concurrently or subsequently treating the subject with RT fractionation.

In an embodiment, an aspect of an embodiment provides, but not limited thereto, a method (or system) wherein the processing may import subject data into the radiation therapy (RT) treatment plan simulation model or access subject data of the radiation therapy (RT) treatment plan simulation model for steps ‘a’ through ‘f’ of said model. Alternatively, the subject data may originate from other sources, devices, systems, or supplies.

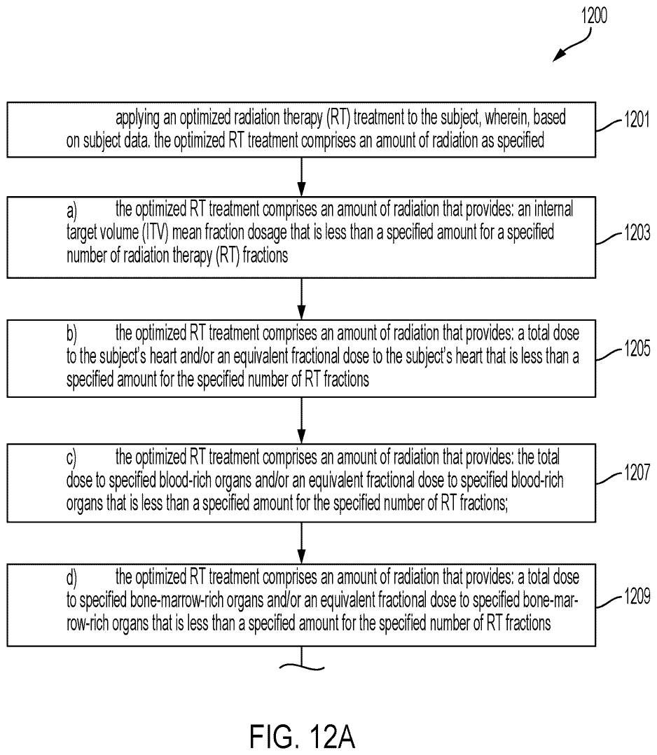

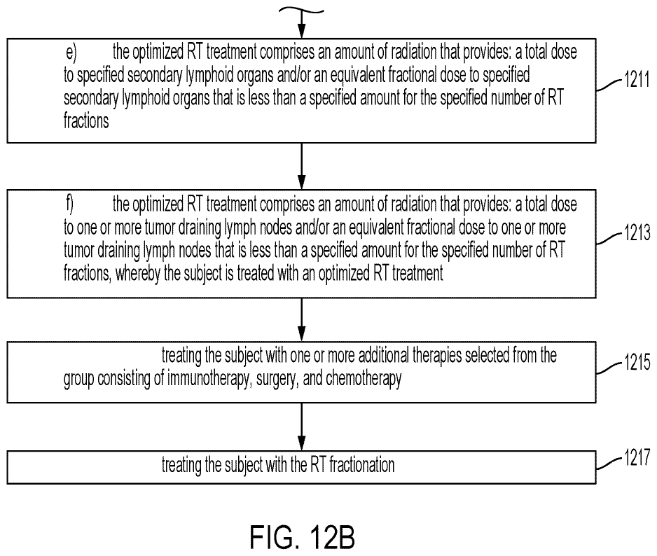

FIGS. 12(A)-(B) is a flow diagram of a method 1200 for treating a subject with an optimizing radiation therapy (RT) treatment. At step 1201, the method comprises applying an optimized radiation therapy (RT) treatment to the subject, based on subject data, such that the optimized RT treatment comprises an amount of radiation as specified. At step 1203, the method includes a) whereby the optimized RT treatment comprises an amount of radiation that provides: an internal target volume (ITV) mean fraction dosage that is less than a specified amount for a specified number of radiation therapy (RT) fractions. At step 1205, the method includes b) whereby the optimized RT treatment comprises an amount of radiation that provides: a total dose to the subject's heart and/or an equivalent fractional dose to the subject's heart that is less than a specified amount for the specified number of RT fractions. At step 1207, the method includes c) whereby the optimized RT treatment comprises an amount of radiation that provides: the total dose to specified blood-rich organs and/or an equivalent fractional dose to specified blood-rich organs that is less than a specified amount for the specified number of RT fractions. At step 1209, the method includes d) whereby the optimized RT treatment comprises an amount of radiation that provides: a total dose to specified bone-marrow-rich organs and/or an equivalent fractional dose to specified bone-marrow-rich organs that is less than a specified amount for the specified number of RT fractions. At step 1211, the method includes e) whereby the optimized RT treatment comprises an amount of radiation that provides: a total dose to specified secondary lymphoid organs and/or an equivalent fractional dose to specified secondary lymphoid organs that is less than a specified amount for the specified number of RT fractions. At step 1213, the method includes f) whereby the optimized RT treatment comprises an amount of radiation that provides: a total dose to one or more tumor draining lymph nodes and/or an equivalent fractional dose to one or more tumor draining lymph nodes that is less than a specified amount for the specified number of RT fractions, whereby the subject is treated with an optimized RT treatment. Moreover, at step 1215, in an embodiment the method may further include treating the subject with one or more additional therapies selected from the group consisting of the immunotherapy, surgery, or chemotherapy. Further yet, at step 1217, in an embodiment the method may further include treating the subject with the RT fractionation.

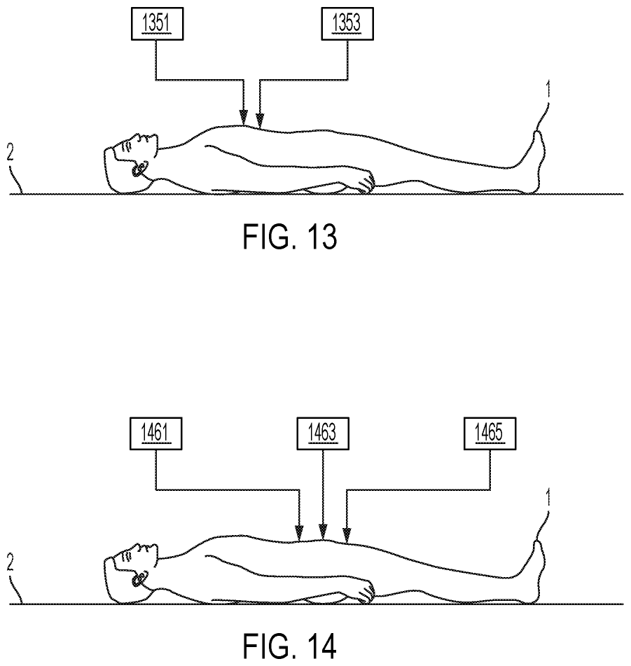

FIG. 13 schematically illustrates a radiation therapy system 1351 treating a subject (or patient) 1 and a molecular imaging system 1353 to image or scan the subject (or patient) 1 such as disposed on the table or surface 2, for example, or the subject 1 may be in an upright position or other desirable position (or multiple positions) suitable for treatment, diagnosis, and therapy.

FIG. 14 schematically illustrates an immunotherapy system 1461, a surgical apparatus, system, instrument or device 1463, or a chemotherapy system 1465 in communication with the subject (or patient) 1 such as disposed on the table or surface 2, for example, or the subject 1 may be in an upright position or other desirable position (or multiple positions) suitable for treatment, diagnosis, and therapy. Any of the systems, apparatuses, devices, or instruments (or portions thereof) of FIGS. 13 and 14 may be combined with one another.

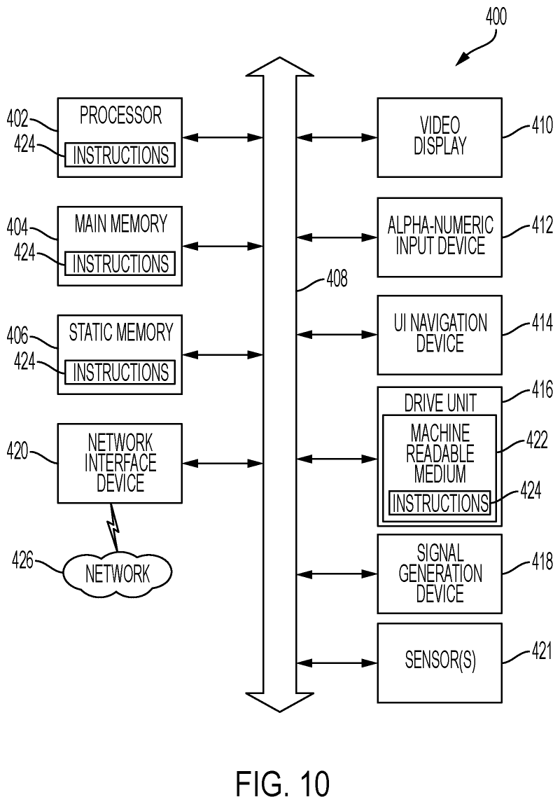

FIG. 10 is a block diagram illustrating an example of a machine upon which one or more aspects of embodiments of the present invention can be implemented.

Referring to FIG. 10, an aspect of an embodiment of the present invention includes, but not limited thereto, a system, method, and computer readable medium that provides: radiation therapy treatment planning criteria or technique to induce tumor reactive T-cells and to reduce radiation induced immune suppression (RIIS) to create an in-situ vaccine against the tumor, which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

### Validity of Model

Some examples and comparisons to the standard arm are given in the following dose distributions discussed below.

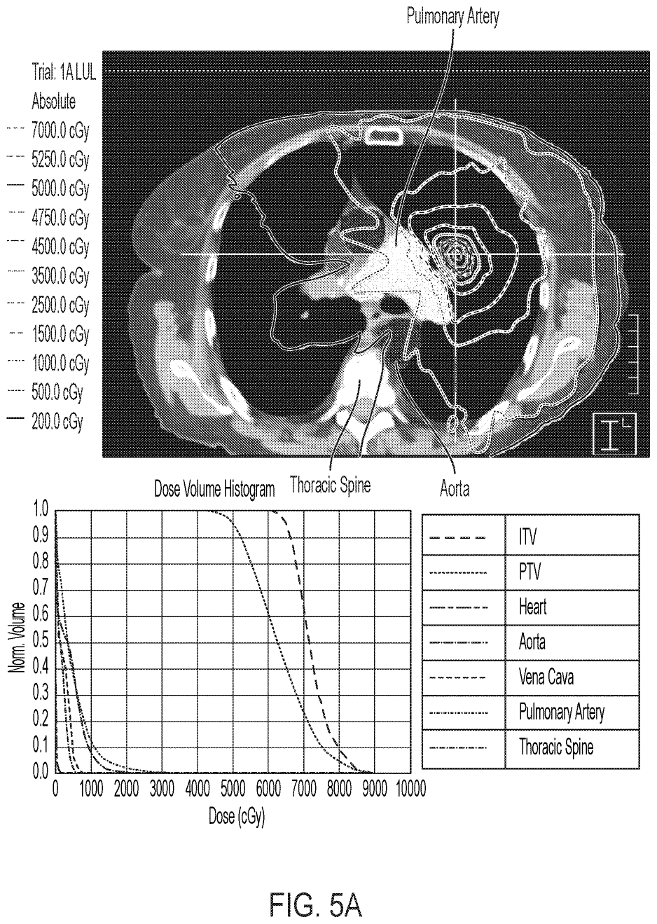

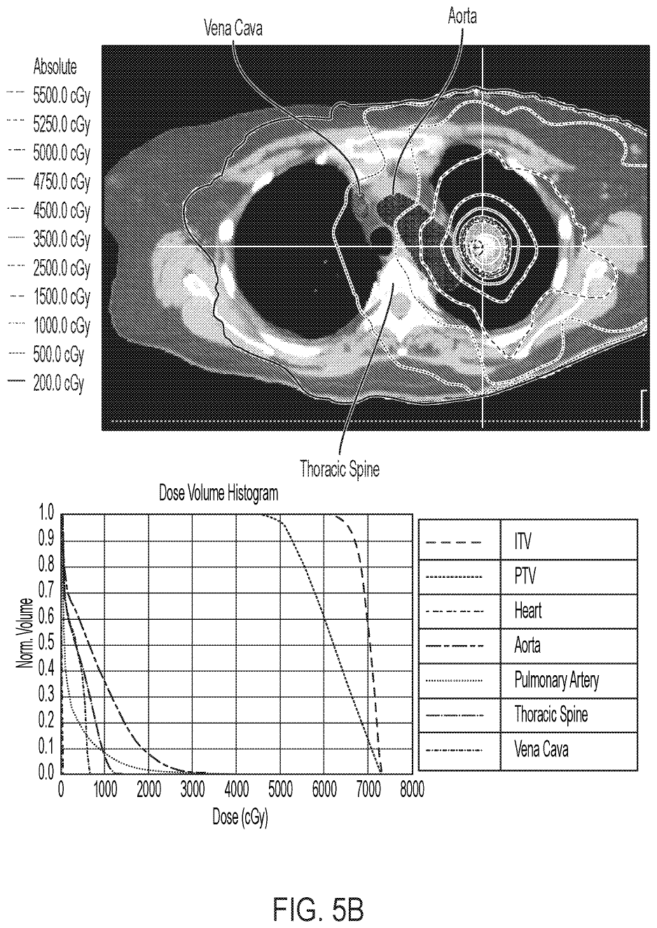

FIG. 5(A), top panel, is a simulated diagram of the superior view [towards the head] from a transverse or axial [horizontal plane dividing top and bottom] cross-section of a subject, with simulated organs showing the inclusion of all organs in the thorax showing the dose distribution for two centrally located tumors; and FIG. 5(A), bottom panel, is graphical representation of the dose volume histogram (DVH). FIGS. 5(A) and 5(B)] are examples of a centrally located tumor where the tumor is close to the blood rich organs, i.e., aorta, pulmonary artery, and vena cava in FIG. 5(A) and tumor close to aorta in the FIG. 5(B). While the top dose distribution (which has met the dosimetric criteria given above for the aorta, vena cava, pulmonary artery and as well as thoracic spine) led to an immune increase via clonal expansion (38%, 45%, and 69% increase from pre-treatment at 5 days, 4 weeks, and 6 months following RT respectively), the bottom dose distribution (which has much higher doses than what was indicated above for aorta and thoracic spine) led to an immune decrease (29% decrease from pre-treatment values at 4 days following RT). Both plans have 60% of the ITV receiving above 70Gy.

Regarding the top panels of FIGS. 5(A), 5(B), 6(A), 6(B), and 6(C) and FIG. 9, as generally illustrated the smaller outline contains higher radiation (cGy) and the larger outline contain lower radiation (cGy). Said differently, as generally illustrated the smaller area of outlined radiation has greater radiation (cGy) and larger area of outlined radiation has lower radiation (cGy).

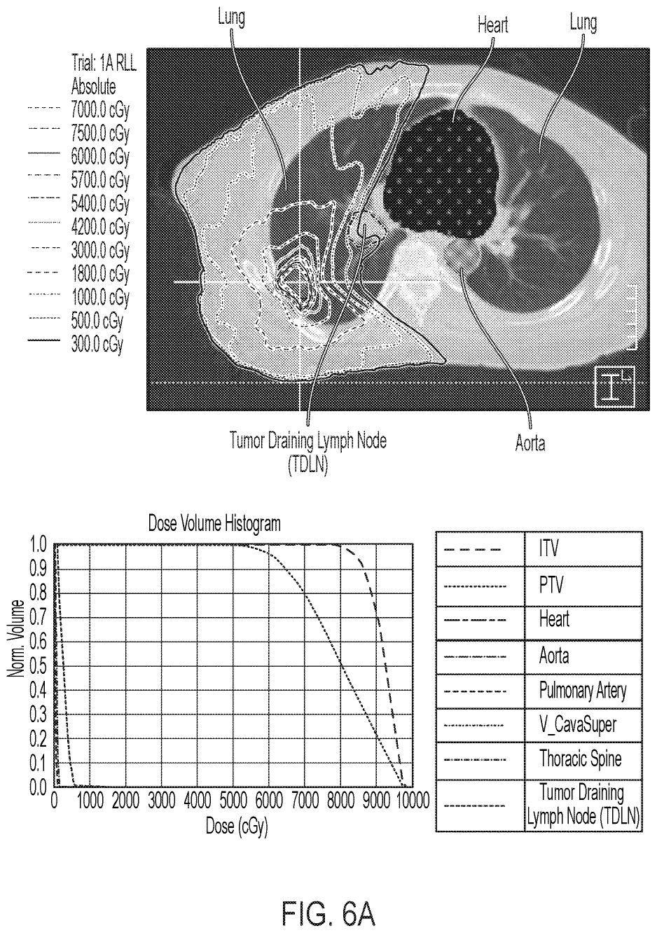

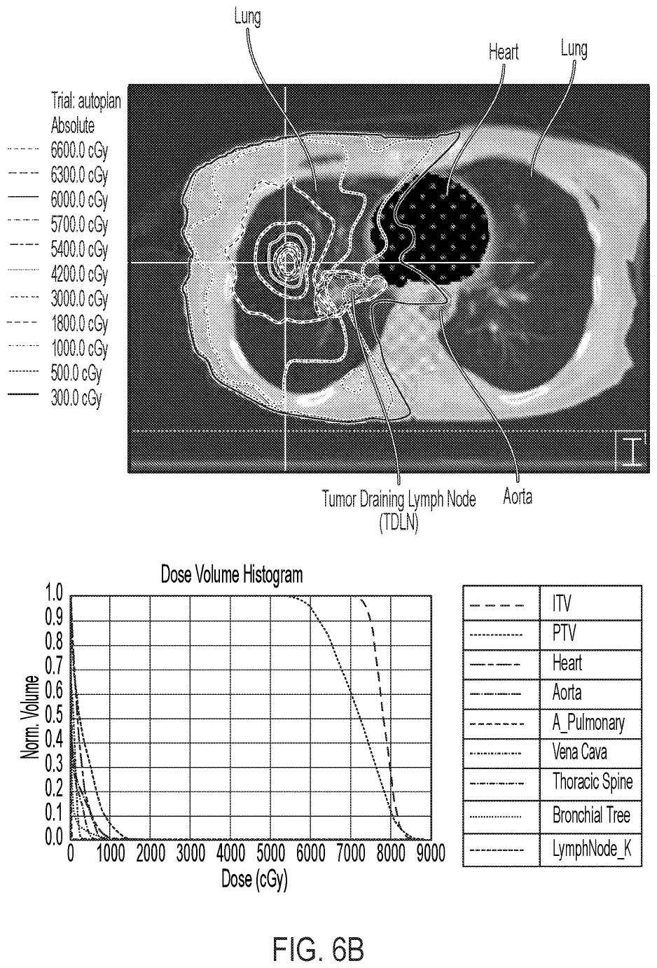

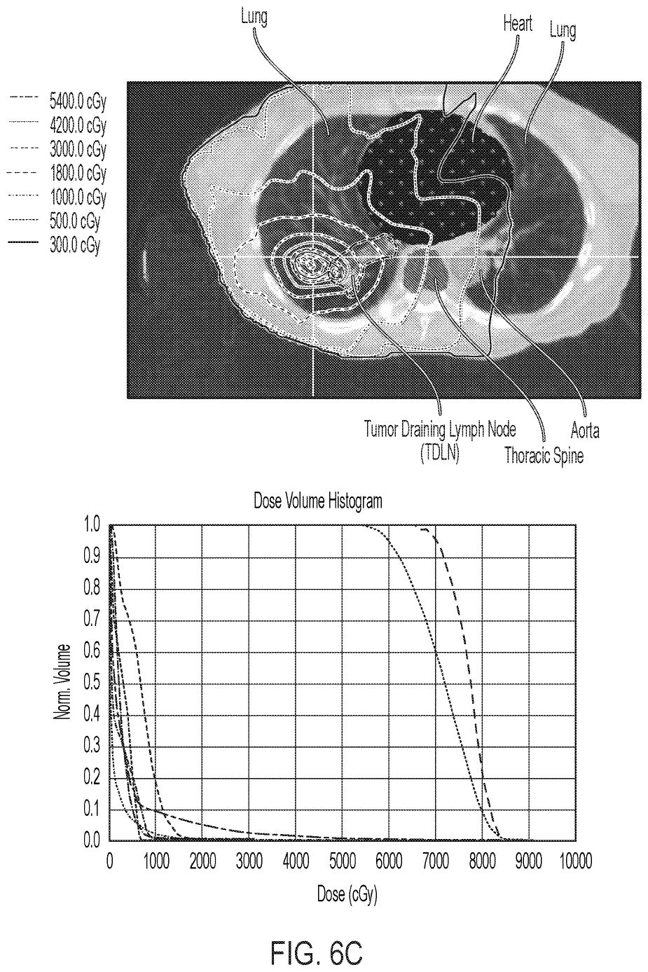

FIG. 6(A), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(A), bottom panel, is graphical representation of the dose volume histogram (DVH).

FIG. 6(B), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(B), bottom panel, is graphical representation of the dose volume histogram (DVH).

FIG. 6(C), top panel, is a simulated diagram of the superior view [towards the head] from a transverse or axial [horizontal plane dividing top and bottom] cross-section of a subject, with simulated organs showing the inclusion of all organs in the thorax showing the dose distribution for three peripherally located tumors; and FIG. 6(C), bottom panel, is graphical representation of the dose volume histogram (DVH).

FIGS. 6(A), 6(B), and 6(C) are examples of a peripherally located tumor where the tumor is close to specific organs. The blood rich organs, e.g., aorta and heart in FIG. 6(A), the thoracic spine in FIG. 6(B), and the tumor draining lymph node in 6(C). While the top two dose distributions (which have met the present dosimetric criteria given above for the aorta, heart, as well as thoracic spine) both reduced RIIS, the top dose distribution had the draining lymph node<200Gy where the clonal expansion could properly occur following the radiation therapy fraction, while the middle dose distribution had a high dose TDLN >500cGy which hindered this process. This led to an immune increase (ALC) via clonal expansion (10%, and 24%, increase from pre-treatment at 4 weeks, and 6 months following RT respectively for the top patient and modest 3% increase from pre-treatment at 5 days for the middle patient). The bottom dose distribution has much higher doses for heart, thoracic spine, and the draining lymph node than what was indicated above, that led to a RIIS (32% decrease from pre-treatment values at 4 months following RT) from pre-treatment values. All three plans have 95%-100% of the ITV receiving a dose between 70-100Gy.

### Preliminary Results:

The present inventor conducted an Institutional Review Board (IRB) approved clinical trial for 50 lung cancer patients treated with SBRT in the absence of chemotherapy between 2019 and 2022, via RTOG 0813 (central) or RTOG 0915 (peripheral), to investigate the ability to reduce Radiation Induced Immune Suppression (RIIS) by reducing dose to blood and lymphatic rich organs. Patients were randomized to two arms: the optimized arm (reduce dose to blood and lymphatic rich organs) and the standard arm. Peripheral blood samples were collected at pre-treatment, at end of treatment, 4 weeks post-treatment, and 6 months post-treatment. No patients with pre-treatment counts less than 0.5×10⁹ cells/L were considered. This radiation treatment plan optimization criteria significantly reduces the dose to circulating blood and lymphatics and minimizes the expected immune cell loss for a given RT plan. Additionally, the present inventor has been able to show that reducing the dose to tumor draining lymph node while maintaining a high dose to the ITV will lead to immune modulation.

TABLE I

| | Difference | | |
|---|---|---|---|
| | (optimized minus | | |
| Time | Standard) | 95% CI | p |
| End of treatment | 14.5% | 0.6%, 28.3% | 0.042 |
| 4 weeks | 15.1% | −1.5%, 31.7% | 0.073. |
| 6 months | 14.7% | −11.8%, 41.3% | 0.267 |

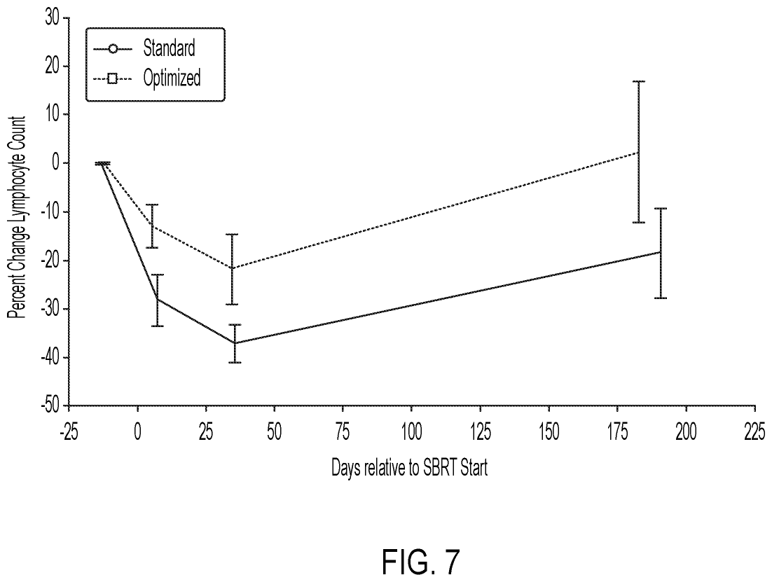

i) Absolute lymphocyte count (ALC) results: Results from the first 33 patients in study are given in Table I as well as in FIG. 7. FIG. 7 is a graphical representation of the preliminary results showing the reduction of immune suppression in the optimized arm at all three time points. Table I provides the percentage difference in absolute lymphocyte counts between the optimized and standard arms for three time points post SBRT stratified for central and peripheral tumor location. The ALC drop (30%) around 1 week post SBRT observed in our standard arm is in agreement with a Japanese clinical trial (72) on lung SBRT (33% reduction, P<0.01), as well as our retrospective data (~30% reduction). Additionally, the present inventor observed a nadir at 4 weeks post SBRT with a 44% lymphocyte reduction in the retrospective data (73) as well as the standard arm of the clinical trial. All three time points are showing a ~15% reduction in RIIS in the optimized arm compared to the standard arm, statistically significant with p=0.042 for the end of treatment point which has the highest amount of data at this point. These results are after adjusting for the stratification factor (74, 75).

The average percentage reductions on integral doses and V5 from the clinically delivered plans are: aorta: 10%, 16.6%; heart: 1.4%, 27.2%; VC: 24%, 43.7%; Thoracic spine: 48.9%, 77.5%; lymph nodes: 26.1%, 45.7%; Total Lung ITV: 0.6%, 0.7%. These lead to a reduction of lymphocyte drop from the clinically delivered plans of 15%. These are very encouraging results showing a direct correlation between the dose to blood and lymph rich organs and the reduction of RIIS. All plans in both arms were performed with 6×-FFF arcs.

ii) Time dependence of ALC: As FIG. 7 shows, the ALC loss at nadir point is 15% less in the optimized arm. Furthermore, the ALC recovery appears to be faster in the optimized arm; on average patients in the optimized arm have recovered fully to their pre-treatment ALC values by 6 months, while this is not the case for standard arm.

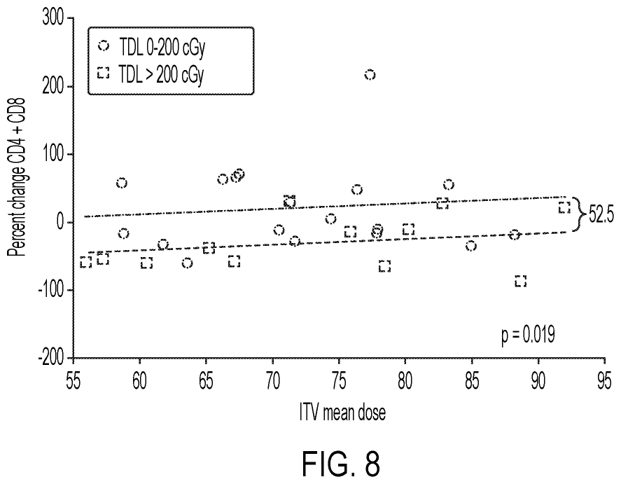

iii) Lymphocyte sub type results: As a second aim, the present inventor measured lymphocyte sub types, CD45+, CD3+, CD4+, CD8+, and Tregs only at the 4 week mark (the present inventor has results for 31/33 patients). The mean (STD) of CD4++CD8+ increase from baseline was only 0.6 (60.7%). However, there are patients with increases as large as 216.7%, and a decrease as low as 86.4% from baseline. FIG. 8 shows the percentage increase of CD4+ and CD8+ cells together as a function of mean dose to ITV. The squares are patients who received Tumor-Draining Lymph Node (TDLN) doses greater than 200Gy. The circles are patients who received TDLN doses less than 200Gy. The two trendlines show a 52.5% increase in the percent change in CD4+ and CD8+ T-cells together after SBRT, adjusting for the ITV mean dose. Note the slight increase of CD4+ and CD8+ cell creation with mean dose to ITV. Of the patients who had an increase in (CD8++CD4+) counts from baseline, all but one had a TDLN dose≤500Gy suggesting that a low TDLN dose is one of the necessary requirements for immune modulation. Even though not statistically significant, the present inventor sees an increase in all measured T-cell sub type numbers at 4 week post treatment, CD3+, CD45+, CD4+ and CD8+ in the optimized arm with respect to the standard arm when adjusted for pre-treatment subtype value, TDLN dose, and ITV volume as shown in Table II. Table II provides the optimized arm T-cell sub type increase with respect to the standard arm when adjusted for pre-treatment subtype value, TDLN dose, and ITV volume. Interestingly, the present inventor only sees this increase when both low dose to TDLN and high dose to the ITV criteria are met. An increase of cytotoxic T-cells could only occur due to clonal expansion. In the literature presented above, there are animal models showing that high dose to tumor encourages dendritic cell maturation, and the other danger signals to occur, that lead the neo-antigen carrying dendritic cell to reach the lymph node where a high density of the naïve T-cells reside and where clonal expansion occur. If the TDLN are irradiated, we risk of killing the naïve T-cells, mature dendritic cells, before the modulation process occurs, as well as the newly created cytotoxic T-cells before they leave the lymph node.

TABLE II

| | Difference (opt − std) | | |
|---|---|---|---|
| Subtype | (cells/microL) | 95% CI | P |
| CD3 | 29.5 | −72, 131 | 0.552 |
| CD45 | 53.2 | −125, 232 | 0.545 |
| CD4 | 13.7 | −40.4, 67.8 | 0.607 |
| CD8 | 7.2 | −90.2, 105 | 0.881 |
| Treg | 3.6 | −16.9, 24.1 | 0.723 |
| CD4 + CD8 | 13.1 | −129, 155 | 0.850 |
| CD3 + CD45 + | 93.9 | −295, 482 | 0.623 |
| CD4 + CD8 | | | |

iv) Reducing dose to blood rich/lymphatic rich OARs lead to reduction in RIIS; the addition of TDLN to OARs increases CD4+ and CD8+ T-cells: Some examples and comparisons of optimized versus standard arm plans are given. All plans were created using Pinnacle treatment planning system (76) per RTOG 0813/0915 guidelines, with VMAT (Volumetric Modulated Arc Therapy) using 6×-FFF beams. The optimized arm plans had doses minimized to blood rich organs (~500cGy) and thoracic spine (~200Gy), on top of the RTOG guidelines, where the standard arms plans followed the RTOG guidelines.

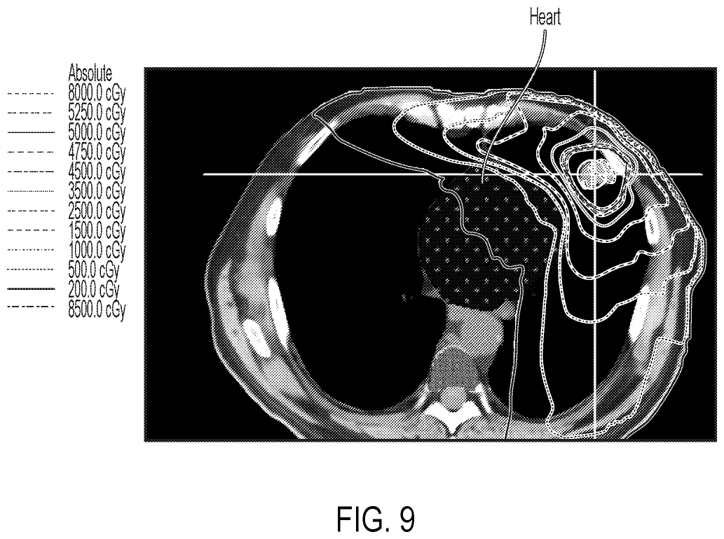

Referring to FIGS. 5(A) and 5(B), top panels, FIGS. 5(A) and 5(B) show examples of centrally located tumors where the tumors are close to the blood rich organs, e.g., aorta, pulmonary artery, and vena cava in the top panel and tumor close to aorta in the bottom panel. While the left dose distribution in the optimized arm had an ALC increase from pre-treatment at 5 days following RT, the right dose distribution (which has much higher doses than what was indicated above for aorta and thoracic spine compared to the optimized plan) led to an ALC decrease (29% decrease from pre-treatment values at 4 days following RT). Both plans have 60% of the ITV (cyan color in DVH) receiving above 70Gy. Both these tumor locations are far from the TDLN so minimal doses to the TDLN received. Referring to FIGS. 6(A), 6(B) and 6(C), top panels, FIGS. 6(A), 6(B), and 6(C) show dose distributions for two peripherally located tumors in the optimized arm of clinical trial. Both tumors are close to the blood rich organs: aorta, and heart and bone marrow rich organ, thoracic spine, and the TDLN. While both dose maps met the dosimetric criteria for the aorta, heart, and thoracic spine, the left patient plan gives a TDLN dose<500cGy, and this patient had a CD4++CD8+ increase of 51.1% from baseline. In contrast, the right patient plan gives a TDLN dose between 1000-1500cGy, and this patient had a CD4++CD8+ decrease of 65.6% (Table III). Table III provides a comparison of TDLN dose, and T-cell sub type increase with respect to baseline for the two patients in FIGS. 6(A), 6(B), and 6(C). Both plans have 95%-100% of the ITV receiving a dose between 70-100Gy. While the aims of the previous clinical trial were simply to reduce immune suppression, the exciting results the present inventor sees for the T-cell sub types and correlation to the TDLN dose provide strong data for this proposal. Another dosimetry example from the clinical trial is given in FIG. 9, where a moderate PTV of 38 cc adjacent to the heart, with thoracic spine and lymphatics getting a very low dose, while the exterior of the heart contour is gracing the 5Gy isodose line. This leads to a plan with minimal doses to blood rich and lymphatic rich organs, and no dose to the TDLN. Referring to FIG. 9, for example, even for a 38 cc PTV close to heart we were able to increase the T-cell sub type percentage from baseline from carefully crafted dose maps. This plan also led to an increase in T-cell sub types at 4 weeks post-treatment. These different examples show that T-cell creation as well as keeping the RIIS down is possible and highly dependent on the proximity to the blood rich/lymphatic rich organs and TDLN. The tools developed in this project could lead to unprecedented improvements in cancer care with no additional cost. The present inventor would like to quantify the best achievable T-cell sub type creation and reduction in immune suppression for sub regions spanning throughout the lung. These tools would be essential for a national clinical trial. Tumor-location-specific, data driven guidelines have to be established, and now can be. Our study has demonstrated that there is significant improvement possible in lung SBRT plans to achieve both RIIS reduction and CTL creation compared to national protocols. Our preliminary study shows that reduction of RIIS as well as creation of CTLs could be achieved by optimizing plans such that the exposure of blood rich/lymph rich organs and the Low Dose Bath (LDB) organs as well as the dose to the TDLN are lower than the above threshold doses. Current clinical protocols do not require TDNL or any lymph rich organ doses to be minimal, or the minimization of the LDB to the healthy tissue surrounding the tumor.

TABLE III

| | Patient top | Patient bottom |
|---|---|---|
| TDLN dose | <500 cGy | 1000-1500 cGy |
| PTV vol (cc) | 20.9 | 12.85 |
| CD3+(% increase) | 10.7 | −27.4 |
| CD45+(% increase) | 9.8 | 10.5 |
| CD4+(% increase) | 20.6 | −54.2 |
| CD8+(% increase) | 30.6 | −86.4 |
| Treg(% increase) | 53.6 | 88.3 |
| CD4+CD8+(% increase) | 51.1 | −65.6 |

### CONCLUSION

An aspect of an embodiment of the present invention provides a system, method, and computer readable medium for, among other things, radiation therapy treatment planning dosimetric criteria that would lead to amelioration of the immune suppression due to radiation, and also lead to the production of tumor infiltrating lymphocytes particular to a given patient's neo-antigens. These criteria will lead to a significant increase of disease-free and overall survival at all stages of cancer. The present inventor's analysis of 38 lung SBRT patients from a clinical trial, where RT treatment plans met all protocol dosimetric criteria, have shown that the dose to the TDLN (first echelon) correlate negatively with T-cell subtype increase. The present inventor also sees a positive correlation between the mean ITV dose and T-cell sub type increase up to doses of approximately 17Gy per fraction. Furthermore, the present inventor has observed the decrease of T-cell creation and the increase of Tregs when the present inventor further increases the dose to the ITV. An aspect of an embodiment of the present invention method, system and computer readable medium is the first to, among other things, perform cytotoxic T-cell creation (CD4+ and CD8+ increase) by manipulation of the critical organs of interest within SBRT in a human clinical trial. Furthermore, an aspect of an embodiment of the present invention method, system, and computer readable medium is the first to, among other things, demonstrate that using treatment planning optimization and a predictive algorithm to reduce the dose to heart and blood rich/lymph rich organs, and also the low dose bath to the whole body, can significantly reduce RIIS.

### EXAMPLES

Practice of an aspect of an embodiment (or embodiments) of the invention will be still more fully understood from the following examples and experimental results, which are presented herein for illustration only and should not be construed as limiting the invention in any way.

Example 1. A computer method for determining a radiation therapy (RT) treatment plan for a subject, said method comprising:
a) determining, using subject data for the subject and a radiation therapy (RT) treatment plan simulation model, an internal target volume (ITV) mean fraction dosage within a first specified amount of radiation dose for a specified number of radiation therapy (RT) fractions;
b) determining, using subject data and the RT treatment plan simulation model, a total dose and/or an equivalent fractional dose to the heart that is less than a second specified amount of radiation;
c) determining, using the subject data and the RT treatment plan simulation model, a total dose and/or an equivalent fractional dose to one or more specified blood-rich organs that is less than a third specified amount of radiation;
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation;
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘f’; and
h) outputting said RT treatment plan for use in concurrently or subsequently treating the subject.

Example 2. The method of example 1, further comprising outputting said RT plan for use in concurrently or subsequently treating the subject with one or more of the following:
immunotherapy, surgery, or chemotherapy.

Example 3. The method of example 1, wherein said radiation therapy (RT) treatment plan is configured for use in concurrently or subsequently treating the subject with RT fractionation.

Example 4. The method of example 3, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

Example 5. The method of example 1, wherein said outputting the RT treatment plan comprises transmitting the RT treatment plan to any one or more of the following:
local memory;
remote memory; or
a display or a graphical user interface.

Example 6. The method of example 1, wherein said processing is accomplished by a computer processor or at least one computer.

Example 7. The method of example 1, wherein said method further comprises:
communicating with a server coupled to a network;
coupling a user interface to said network; and
coupling an application to said server and/or said user interface.

Example 8. The method of example 1, wherein said subject data comprises any one or more of the following:
a) anatomic imaging CT, MRI, ultrasound, or planar imaging,
b) functional imaging,
c) molecular imaging and optical imaging,
d) nuclear imaging,
e) pre-treatment immune cell levels (for example, but not limited thereto, sub types and functional),
f) structure set, and
g) Immune cells (wherein the functional imaging may include for example, but not limited thereto, PET or MRI pulse sequences).

Example 9. The method of example 8, wherein said structure set comprises any one or more of the following:
a) gross target volume (GTV);
b) clinical Target Volume (CTV);
c) internal Target Volume (ITV);
d) planning Target Volume (PTV);
e) location of the tumor;
f) blood rich organs;
g) lymph rich organs;
h) primary lymphoid organs (for example, but not limited thereto, wherein immune cells originate);
i) secondary lymphoid organs (for example, but not limited thereto, wherein immune cells accumulate as well as the immune modulation takes place); or
j) tumor draining lymph node (for example, but not limited thereto, wherein the occurrence of immune modulation is highest).

Example 10. The method of example 8, wherein said immune cells comprise any one or more of the following (and for example, but not limited thereto, wherein all of which will have a reduced levels of cell kill, while some types including CD4+ and CD8+ increase in numbers due to immune modulation due to the optimization):
a) Lymphocytes;
b) CD4+ (aka Helper T-Cells);
c) CD8+ (aka Cytotoxic T-Cells, or Killer T-Cells);
d) Tregs (aka Regulatory T-Cells);
e) CD45+;
f) CD3+;
g) CD19+;
h) CD56+;
i) Naïve T and B cells;
j) Activated T and B cells;
k) Memory T and B cells;
l) Cytotoxic T cells;
m) B cells;
n) Natural Killer cells;
o) Dendritic cells;
p) Neutrophils;
q) Macrophages;
r) Monocytes;
s) mast cells; and
t) Eosinophil.

Example 11. The method of example 1, wherein said subject data comprises any one or more of the following patient factors:
a) subject age;
b) blood cell sub-type distribution;
c) pre-treatment rate of regeneration;
d) pre-treatment rate of redistribution;
e) type of radiation; or
f) molecular imaging.

Example 12. The method of example 1, wherein:
said keeping an internal target volume (ITV) mean fraction dosage within said specified amount of radiation is within the range of about 14Gy to about 17Gy for said specified number of RT fractions.

13. The method of example 1, wherein:
said keeping the total dose to heart to less than said specified amount of radiation is about 5Gy.

Example 14. The method of example 1, wherein:
said keeping the equivalent fractional dose to the heart to less than said specified amount of radiation is about 5Gy/N where N is number of fractions. ‘N’ may be five, or may be less than or greater than five. In an embodiment, ‘N’ may be in the range of greater than zero and less than 50; or may be greater than 50.

In an embodiment, said keeping the equivalent fractional dose to heart to less than said specified amount of radiation is determined by a blood circulation model.

Example 15. The method of example 1, wherein:
said specified blood-rich organs comprise one or more of the following: aorta, vena cava, pulmonary artery, liver, hepatic artery, and brain.

Example 16. The method of example 1, wherein:
said keeping the total dose to specified blood-rich organs to less than said specified amount of radiation is about 8Gy. In an embodiment, the specified amount of radiation may be less than or greater than 8Gy.

Example 17. The method of example 1, wherein:
said keeping the equivalent fractional dose to specified blood-rich organs to less than said specified amount of radiation is 8Gy/N where N is number of fractions. ‘N’ may be eight, or may be less than or greater than eight. In an embodiment, ‘N’ may be in the range of greater than zero and less than 80; or may be greater than 80.

In an embodiment said keeping the equivalent fractional dose to specified blood-rich organs to less than said specified amount of radiation is determined by a blood circulation model.

Example 18. The method of example 1, wherein:
said specified bone-marrow-rich organs comprises any one or more of the following: spine, the pelvic bone, and the thigh bone.

Example 19. The method of example 1, wherein:
said keeping the total dose to specified bone-marrow-rich organs to less than said specified amount of radiation is about 2Gy. In an embodiment, the specified amount of radiation may be less than or greater than 2Gy.

Example 20. The method of example 1, wherein:
said keeping the equivalent fractional dose to specified bone-marrow-rich organs to less than said specified amount of radiation is about 40cGy. In an embodiment, the specified amount of radiation may be less than or greater than 40cGy.

Example 21. The method of example 1, wherein:
said secondary lymphoid organs comprise any one more of the following: lymph nodes, spleen, tonsils, Peyer's Patches, adenoids, nasal associated lymphoid tissues, and mucosal tissues.

Example 22. The method of example 1, wherein:
said keeping the total dose to specified secondary lymphoid organs to less than said specified amount of radiation is about 2Gy. In an embodiment, the specified amount of radiation may be less than or greater than 2Gy.

Example 23. The method of example 1, wherein:
said keeping the equivalent fractional dose to specified secondary lymphoid organs to less than said specified amount of radiation is about 40cGy. In an embodiment, the specified amount of radiation may be less or greater than 40cGy.

Example 24. The method of example 1, wherein:
said keeping the total dose to tumor draining lymph node to less than said specified amount of radiation is about 2Gy. In an embodiment, the specified amount of radiation may be less than or greater than 2Gy.

Example 25. The method of example 1, wherein:
said keeping the equivalent fractional dose to tumor draining lymph node to less than said specified amount of radiation is about 40cGy. In an embodiment, the specified amount of radiation may be less than or greater than 40cGy.

Example 26. The method of example 1, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

Example 27. The method of example 1, wherein delivery of said radiation therapy (RT) treatment plan comprises any one or more of the following:
a) three-dimensional conformal radiation therapy (3DCRT) fractions;
b) intensity-modulated radiation therapy (IMRT) fractions;
c) volumetric modulated arc radiation therapy (VMAT) fractions; and
d) Rapid Arc fractions.

Example 28. The method of example 1, wherein said radiation therapy (RT) plan comprises molecular imaging. For example, wherein molecular imaging may include, but not limited thereto, any one or more of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT).

Example 29. The method of example 1, wherein said subject patient data comprises any one or more of the following patient/tumor microenvironment factors that could determine the required mean fraction dosage to the internal target volume (ITV) which could be extracted from personalized medicine via artificial intelligence or machine learning:
a) subject age;
b) pre-Tx blood cell sub-type distribution;
c) amount of circulating tumor DNA (ct-DNA);
d) tumor PD-1, and PD-L1 expression;
e) tumor burden;
f) tumor type and stage;
g) type of radiation;
h) cytokines and cell surface markers in the tumor microenvironment;
i) proteomics;
j) radiomics; or
k) genomics.

Example 30. A system for use in determining a radiation therapy (RT) treatment plan for a subject, said system comprising:
a computer processor;
a memory configured to store instructions that are executable by said computer processor, wherein said computer processor is configured to execute the instructions for:
a) determining, using subject data for the subject and a radiation therapy (RT) treatment plan simulation model, an internal target volume (ITV) mean fraction dosage within a specified amount of radiation dose for a first specified number of radiation therapy RT fractions;
b) determining, using the subject data and the RT treatment plan simulation model, a total dose and/or an equivalent fractional dose to the heart that is less than a second specified amount of radiation
c) determining, using the subject data and the RT treatment plan simulation model, a total dose and/or an equivalent fractional dose to one or more specified blood-rich organs that is less than a third specified amount of radiation
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘; and
h) outputting said RT treatment plan for use in concurrently or subsequently treating the subject.

Example 31. The system of example 30, wherein said system further comprises outputting said RT treatment plan for use in concurrently or subsequently treating the subject with one or more of the following:
immunotherapy, surgery, or chemotherapy.

Example 32. The system of example 33, wherein said radiation therapy (RT) treatment plan is configured for use in concurrently or subsequently treating the subject with RT fractionation.

Example 33. The system of example 32, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

Example 34. The system of example 30, wherein said outputting the RT treatment plan comprises transmitting the RT treatment plan to any one or more of the following:
local memory;
remote memory; or
a display or a graphical user interface.

Example 35. The system of example 30, wherein said processing is accomplished by a computer processor or at least one computer.

Example 36. The system of example 30, wherein said system further comprises:
communicating with a server coupled to a network;
coupling a user interface to said network; and
coupling an application to said server and/or said user interface.

Example 37. The system of example 30, wherein said memory comprises a main memory and a static memory.

Example 38. The system of example 30, wherein said memory comprises one or more of any one of the following:
electrically programmable read-only memory;
electrically erasable programmable read-only memory;
flash memory drive;
magnetic disk;
internal hard disk;
external hard disk;
solid-state drive;
removable disk;
magneto-optical disk;
CD-ROM disk; or
DVD-ROM disk.

Example 39. The system of example 30, wherein said subject data comprises any one or more of the following:
a) anatomic imaging CT, MRI, ultrasound, or planar imaging,
b) functional imaging,
c) molecular imaging and optical imaging,
d) nuclear imaging,
e) pre-treatment immune cell levels (for example, but not limited thereto, absolute, sub types, and functional),
f) structure set, and
g) Immune cells (wherein the functional imaging may include for example, but not limited thereto, PET or MRI pulse sequences).

Example 40. The system of example 39, wherein said structure set comprises any one or more of the following:
a) gross target volume (GTV);
b) clinical Target Volume (CTV);
c) internal Target Volume (ITV);
d) planning Target Volume (PTV);
e) location of the tumor;
f) blood rich organs;
g) lymph rich organs;
h) primary lymphoid organs (for example, but not limited thereto, wherein immune cells originate);
i) secondary lymphoid organs (for example, but not limited thereto, wherein immune cells accumulate as well as the immune modulation takes place); or
j) tumor draining lymph node (for example, but not limited thereto, wherein the occurrence of immune modulation is highest).

Example 41. The system of example 39, wherein said immune cells comprise any one or more of the following (for example, but not limited thereto, wherein all of which will have a reduced levels of cell kill, while some types including CD4+ and CD8+ increase in numbers due to immune modulation due to the optimization):
a) Lymphocytes;
b) CD4+ (aka Helper T-Cells);
c) CD8+ (aka Cytotoxic T-Cells, or Killer T-Cells);
d) Tregs (aka Regulatory T-Cells);
e) CD45+;
f) CD3+;
g) CD19+;
h) CD56+;
i) Naïve T and B cells;
j) Activated T and B cells;
k) Memory T and B cells;
l) Cytotoxic T cells;
m) B cells;
n) Natural Killer cells;
o) Dendritic cells;
p) Neutrophils;
q) Macrophages;
r) Monocytes;
s) mast cells; and
t) Eosinophil.

Example 42. The system of example 30, wherein said subject data comprises any one or more of the following patient factors:
a) subject age;
b) blood cell sub-type distribution;
c) pre-treatment rate of regeneration;
d) pre-treatment rate of redistribution;
e) type of radiation; or
f) molecular imaging.

Example 43. The system of example 30, wherein:
said keeping an internal target volume (ITV) mean fraction dosage within said specified amount of radiation is within the range of about 14Gy to about 17Gy for said specified number of RT fractions.

Example 44. The system of example 30, wherein:
said keeping the total dose to heart to less than said specified amount of radiation is about 5Gy.

Example 45. The system of example 30, wherein:
said keeping the equivalent fractional dose to the heart to less than said specified amount of radiation is about 5Gy/N where N is number of fractions.

46. The system of example 30, wherein:
said specified blood-rich organs comprise one or more of the following: aorta, vena cava, pulmonary artery, liver, hepatic artery, and brain.

Example 47. The system of example 30, wherein:
said keeping the total dose to specified blood-rich organs to less than said specified amount of radiation is about 8Gy.

Example 48. The system of example 30, wherein:
said keeping the equivalent fractional dose to specified blood-rich organs to less than said specified amount of radiation is 8Gy/N where N is number of fractions.

Example 49. The system of example 30, wherein:
said specified bone-marrow-rich organs comprises any one or more of the following: spine, the pelvic bone, and the thigh bone.

Example 50. The system of example 30, wherein:
said keeping the total dose to specified bone-marrow-rich organs to less than said specified amount of radiation is about 2Gy.

Example 51. The system of example 30, wherein:
said keeping the equivalent fractional dose to specified bone-marrow-rich organs to less than said specified amount of radiation is about 40cGy.

Example 52. The system of example 30, wherein:
said secondary lymphoid organs comprise any one more of the following: lymph nodes, spleen, tonsils, Peyer's Patches, adenoids, nasal associated lymphoid tissues, and mucosal tissues.

Example 53. The system of example 30, wherein:
said keeping the total dose to specified secondary lymphoid organs to less than said specified amount of radiation is about 2Gy.

Example 54. The system of example 30, wherein:
said keeping the equivalent fractional dose to specified secondary lymphoid organs to less than said specified amount of radiation is about 40cGy.

Example 55. The system of example 30, wherein:
said keeping the total dose to tumor draining lymph node to less than said specified amount of radiation is about 2Gy.

Example 56. The system of example 30, wherein:
said keeping the equivalent fractional dose to tumor draining lymph node to less than said specified amount of radiation is about 40cGy.

Example 57. The system of example 30, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

Example 58. The system of example 30, wherein delivery of said radiation therapy (RT) treatment plan comprises any one or more of the following:
a) three-dimensional conformal radiation therapy (3DCRT) fractions;
b) intensity-modulated radiation therapy (IMRT) fractions;
c) volumetric modulated arc radiation therapy (VMAT) fractions; and
d) Rapid Arc fractions.

Example 59. The system of example 30, wherein said radiation therapy (RT) plan comprises molecular imaging. For example, wherein molecular imaging may include, but not limited thereto, any one or more of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT).

Example 60. The system of example 30, wherein said subject patient data comprises any one or more of the following patient/tumor microenvironment factors that could determine the required mean fraction dosage to the internal target volume (ITV) which could be extracted from personalized medicine via artificial intelligence or machine learning:
a) subject age;
b) pre-Tx blood cell sub-type distribution;
c) amount of circulating tumor DNA (ct-DNA);
d) tumor PD-1, and PD-L1 expression;
e) tumor burden;
f) tumor type and stage;
g) type of radiation;
h) cytokines and cell surface markers in the tumor microenvironment;
i) proteomics;
j) radiomics; or
k) genomics.

Example 61. A non-transitory, computer readable storage medium having instructions stored thereon for use in determining a radiation therapy (RT) treatment plan for a subject, that, when executed by a computer processor, cause the computer processor to:
a) determine, using subject data for the subject and a radiation therapy (RT) treatment plan simulation model, an internal target volume (ITV) mean fraction dosage within a specified amount of radiation dose for a first specified number of radiation therapy (RT) fractions;
b) determine, using the subject data and the RT treatment plan simulation model, a total dose and/or an equivalent fractional dose to the heart that is less than a second specified amount of radiation
c) determine, using the subject data and the RT treatment plan simulation model, a total dose and/or an equivalent fractional dose to one or more specified blood-rich organs that is less than a third specified amount of radiation
d) determine, using the subject data and the RT treatment plan simulation model, a total dose and/or an equivalent fractional dose to one or more specified bone-marrow-rich organs that is less than a fourth specified amount of radiation
e) determine, using the subject data and the RT treatment plan simulation model, a total dose and/or an equivalent fractional dose to one or more specified secondary lymphoid organs that is less than a fifth specified amount of radiation
f) determine, using the subject data and the RT treatment plan simulation model, a total dose and/or an equivalent fractional dose to a tumor draining lymph node that is less than a sixth specified amount of radiation;
g) determine the radiation therapy (RT) treatment plan for the subject using results from the processing steps ‘a’ through ‘f’; and
h) output said RT treatment plan for use in concurrently or subsequently treating the subject.

Example 62. The computer readable storage medium of example 61, wherein, when executed by the computer processor, causes the computer processor to output said RT treatment plan for use in concurrently or subsequently treating the subject with one or more of the following:
immunotherapy, surgery, or chemotherapy.

Example 63. The computer readable storage medium of example 61, wherein, said radiation therapy (RT) treatment plan is configured for use in concurrently or subsequently treating the subject with RT fractionation.

Example 64. The computer readable storage medium of example 63, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

Example 65. The computer readable storage medium of example 61, wherein said outputting the RT treatment plan comprises transmitting the RT treatment plan to any one or more of the following:
local memory;
remote memory; or
a display or a graphical user interface.

Example 66. The computer readable storage medium of example 61, wherein said computer processor comprises at least one computer.

Example 67. The computer readable storage medium of example 61, wherein, when executed by the computer processor, causes the computer processor to communicate with:
a server coupled to a network;
a user interface to said network; and
an application coupled to said server and/or said user interface.

Example 68. The computer readable storage medium of example 61, wherein said subject data comprises any one or more of the following:
a) anatomic imaging CT, MRI, ultrasound, or planar imaging,
b) functional imaging,
c) molecular imaging and optical imaging,
d) nuclear imaging,
e) pre-treatment immune cell levels,
f) structure set, and
g) Immune cells.

Example 69. The computer readable storage medium of example 68, wherein said structure set comprises any one or more of the following:
a) gross target volume (GTV);
b) clinical Target Volume (CTV);
c) internal Target Volume (ITV);
d) planning Target Volume (PTV);
e) location of the tumor;
f) blood rich organs;
g) lymph rich organs;
h) primary lymphoid organs;
i) secondary lymphoid organs; or
j) tumor draining lymph node.

Example 70. The computer readable storage medium of example 68, wherein said immune cells comprise any one or more of the following:
a) Lymphocytes;
b) CD4+ (aka Helper T-Cells);
c) CD8+ (aka Cytotoxic T-Cells, or Killer T-Cells);
d) Tregs (aka Regulatory T-Cells);
e) CD45+;
f) CD3+;
g) CD19+;
h) CD56+;
i) Naïve T and B cells;
j) Activated T and B cells;
k) Memory T and B cells;
l) Cytotoxic T cells;
m) B cells;
n) Natural Killer cells;
o) Dendritic cells;
p) Neutrophils;
q) Macrophages;
r) Monocytes;
s) mast cells; and
t) Eosinophil.

Example 71. The computer readable storage medium of example 61, wherein said subject data comprises any one or more of the following patient factors:
a) subject age;
b) blood cell sub-type distribution;
c) pre-treatment rate of regeneration;
d) pre-treatment rate of redistribution;
e) type of radiation; or
f) molecular imaging.

Example 72. The computer readable storage medium of example 61, wherein:
said keeping an internal target volume (ITV) mean fraction dosage within said specified amount of radiation is within the range of about 14Gy to about 17Gy for said specified number of RT fractions.

Example 73. The computer readable storage medium of example 61, wherein:
said keeping the total dose to heart to less than said specified amount of radiation is about 5Gy.

Example 74. The computer readable storage medium of example 61, wherein:
said keeping the equivalent fractional dose to the heart to less than said specified amount of radiation is about 5Gy/N where N is number of fractions.

Example 75. The computer readable storage medium of example 61, wherein:
said specified blood-rich organs comprise one or more of the following: aorta, vena cava, pulmonary artery, liver, hepatic artery, and brain.

Example 76. The computer readable storage medium of example 61, wherein:
said keeping the total dose to specified blood-rich organs to less than said specified amount of radiation is about 8Gy.

Example 77. The computer readable storage medium of example 61, wherein:
said keeping the equivalent fractional dose to specified blood-rich organs to less than said specified amount of radiation is 8Gy/N where N is number of fractions.

Example 78. The computer readable storage medium of example 61, wherein:
said specified bone-marrow-rich organs comprises any one or more of the following: spine, the pelvic bone, and the thigh bone.

Example 79. The computer readable storage medium of example 61, wherein:
said keeping the total dose to specified bone-marrow-rich organs to less than said specified amount of radiation is about 2Gy.

Example 80. The computer readable storage medium of example 61, wherein:
said keeping the equivalent fractional dose to specified bone-marrow-rich organs to less than said specified amount of radiation is about 40cGy.

Example 81. The computer readable storage medium of example 61, wherein:
said secondary lymphoid organs comprise any one more of the following: lymph nodes, spleen, tonsils, Peyer's Patches, adenoids, nasal associated lymphoid tissues, and mucosal tissues.

Example 82. The computer readable storage medium of example 61, wherein:
said keeping the total dose to specified secondary lymphoid organs to less than said specified amount of radiation is about 2Gy.

Example 83. The computer readable storage medium of example 61, wherein:
said keeping the equivalent fractional dose to specified secondary lymphoid organs to less than said specified amount of radiation is about 40cGy.

Example 84. The computer readable storage medium of example 61, wherein:
said keeping the total dose to tumor draining lymph node to less than said specified amount of radiation is about 2Gy.

Example 85. The computer readable storage medium of example 61, wherein:
said keeping the equivalent fractional dose to tumor draining lymph node to less than said specified amount of radiation is about 40cGy.

Example 86. The computer readable storage medium of example 61, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

Example 87. The computer readable storage medium of example 61, wherein delivery of said radiation therapy (RT) treatment plan comprises any one or more of the following:
a) three-dimensional conformal radiation therapy (3DCRT) fractions;
b) intensity-modulated radiation therapy (IMRT) fractions;
c) volumetric modulated arc radiation therapy (VMAT) fractions; and
d) Rapid Arc fractions.

Example 88. The computer readable storage medium of example 61, wherein said radiation therapy (RT) plan comprises molecular imaging.

Example 89. The computer readable storage medium of example 61, wherein said subject patient data comprises any one or more of the following patient/tumor microenvironment factors that could determine the required mean fraction dosage to the internal target volume (ITV) which could be extracted from personalized medicine via artificial intelligence or machine learning:
a) subject age;
b) pre-Tx blood cell sub-type distribution;
c) amount of circulating tumor DNA (ct-DNA);
d) tumor PD-1, and PD-L1 expression;
e) tumor burden;
f) tumor type and stage;
g) type of radiation;
h) cytokines and cell surface markers in the tumor microenvironment;
i) proteomics;
j) radiomics; or
k) genomics.

Example 90. A method for treating a subject with an optimizing radiation therapy (RT) treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
whereby the subject is treated with an optimized RT treatment.

Example 91. The method according to example 90, further comprising treating the subject with one or more additional therapies selected from the group consisting of immunotherapy, surgery, and chemotherapy.

Example 92. The method according to example 91, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

Example 93. The method according to example 90, further comprising treating the subject with RT fractionation.

Example 94. The method accordingly to any one of examples 90-93, wherein:
(i) the internal target volume (ITV) mean fraction dosage is about 14 to about 17 Gy per fraction; and/or
(ii) the total dose to the subject's heart, aorta, vena cava, and pulmonary artery is less than about 8 Gy, optionally less than about 5 Gy; and/or
(iii) the total dose to the subject's thoracic spine is less than about 2 Gy; and/or
(iv) the total dose to the subject's lymph nodes and/or lymphatics is less than about 2 Gy; and/or
(v) the total dose to the subject's one or more tumor draining lymph nodes is less than about 2 Gy.

Example 95. A method for reducing the risk of developing Treatment Related Lymphopenia (TRL) and/or radiation induced immune suppression (RIIS) in a subject undergoing a cancer and/or a tumor treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment reduces the risk of developing TRL and/or RIIS in the subject relative to a standard RT treatment.

Example 96. The method of example 95, wherein the subject has a cancer and/or a tumor selected from the group consisting of glioblastoma, advanced stage non-small cell lung cancer (NSCLC), pancreatic cancer, and squamous cell carcinoma of the head and neck.

Example 97. The method of example 94 or example 95, wherein the optimized RT treatment increases survival of the subject relative to a standard RT treatment.

Example 98. The method according to any one of examples 95-97, further comprising treating the subject with one or more additional therapies selected from the group consisting of immunotherapy, surgery, and chemotherapy.

Example 99. The method according to example 98, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

Example 100. A method for inducing tumor reactive T cells in a subject undergoing an anti-tumor treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment induces tumor reactive T cells in the subject.

Example 101. The method according to example 100, further comprising treating the subject with one or more additional therapies selected from the group consisting of immunotherapy, surgery, and chemotherapy.

Example 102. The method according to example 101, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

Example 103. Use of an optimized radiation therapy (RT) treatment for treating a subject with a disease, disorder, or condition for which radiation therapy would be appropriate, and/or for reducing the risk of developing Treatment Related Lymphopenia (TRL) and/or radiation induced immune suppression (RIIS), and/or for inducing reactive T cells, wherein the optimized radiation therapy (RT) treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
and further wherein the optimized radiation therapy (RT) treatment treats the disease, disorder, or condition; reduces the risk of developing Treatment Related Lymphopenia (TRL) and/or radiation induced immune suppression (RIIS); and/or induces reactive T cells in the subject.

Example 104. The use according to example 103, wherein the subject has a cancer and/or a tumor selected from the group consisting of glioblastoma, advanced stage non-small cell lung cancer (NSCLC), pancreatic cancer, and squamous cell carcinoma of the head and neck.

Example 105. The use according to example 102 or example 103, wherein the optimized RT treatment increases survival of the subject relative to a standard RT treatment.

Example 106. The use according to any one of examples 103-105, further comprising treating the subject with one or more additional therapies selected from the group consisting of immunotherapy, surgery, and chemotherapy.

Example 107. The use according to example 106, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof. CL REFERENCES

The devices, systems, apparatuses, modules, compositions, materials, computer program products, non-transitory computer readable medium, and methods of various embodiments of the invention disclosed herein may utilize aspects (such as devices, apparatuses, modules, systems, compositions, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
1. Lee Y, Auh S L, Wang Y, et al. Therapeutic effects of ablative radiation on local tumor require CD8+ T-cells: changing strategies for cancer treatment. Blood 2009; 114:589-95.
2. Schaue D, Ratikan J A, Iwamoto K S, McBride W H. Maximizing tumor immunity with fractionated radiation. Int J Radiat Oncol Biol Phys 2012; 83:1306-10.
3. Grutters J P C, Kessels A G H, Pijls-Johannesma M et al. Comparison of the effectiveness of radiotherapy with photons, protons and carbon-ions for non-small cell lung cancer: A meta-analysis. Radiotherapy and Oncology 95 (2010) 32-40.
4. McMullen T., Liyanage S., Alonso, C. W K. Radiation Related Lymphopenia for Lung SBRT Treatments: Measurement Versus Simulation. Med Phys. 2017; 44(6):3118.
5. NIH-1R01CA234281-01A1: Early Phase Clinical Trials in Imaging and Image-Guided Interventions: “Ameliorating lymphocyte depletion and functional impairment following lung SBRT”
6. Goldstraw P, Crowley J, Chansky K, et al. The IASLC Lung Cancer Staging Project: proposals for the revision of the TNM stage groupings in the forthcoming (seventh) edition of the TNM Classification of malignant tumours. J Thorac Oncol 2007; 2:706-14.
7. Shohan J. Some theoretical considerations on the present status of roentgen therapy. N Engl J Med. 1916; 175:321-327.
8. Maclennan I C M, Kay H E M. Analysis of treatment in childhood leukemia. IV. The critical association between dose fractionation and immunosuppression induced by cranial irradiation. Cancer. 1978; 41(1):108-11.
9. Weeke E. The development of lymphopenia in uremic patients undergoing extracorporeal irradiation of the blood with portable beta units. Radiat Res. 1973; 56(3):554-9.
10. Grossman S A, Ye X, Lesser G, Sloan A, Carraway H, Desideri S, et al. Immunosuppression in patients with high-grade gliomas treated with radiation and temozolomide. Clin Cancer Res. 2011; 17(16):5473-80.
11. Campian J L, Ye X, Brock M, Grossman S A. Treatment-related lymphopenia in patients with stage III non-small-cell lung cancer. Cancer Invest. 2013; 31(3):183-8.
12. Balmanoukian A, Ye X, Herman J, Laheru D, Grossman S A. The association between treatment-related lymphopenia and survival in newly diagnosed patients with resected adenocarcinoma of the pancreas. Cancer Invest. 2012; 30(8):571-6.
13. Wild A T, Ye X, Ellsworth S G, Smith J A, Narang A K, Garg T, et al. The Association Between Chemoradiation-related Lymphopenia and Clinical Outcomes in Patients With Locally Advanced Pancreatic Adenocarcinoma. Am J Clin Oncol. 2015; 38(3):259-65.
14. Campian J L, Sarai G, Ye X, Marur S, Grossman S A. Association between severe treatment-related lymphopenia and progression-free survival in patients with newly diagnosed squamous cell head and neck cancer. Head Neck. 2014; 36(12):1747-53.
15. Grossman S A, Ellsworth S, Campian J, Wild A T, Herman J M, Laheru D, et al. Survival in Patients With Severe Lymphopenia Following Treatment With Radiation and Chemotherapy for Newly Diagnosed Solid Tumors. J Natl Compr Canc Netw. 2015; 13(10):1225-31.
16. Hughes M A, Parisi M, Grossman S, Kleinberg L. Primary brain tumors treated with steroids and radiotherapy: Low CD4 counts and risk of infection. Int J Radiat Oncol Biol Phys. 2005; 62(5):1423-6.
17. Kaplan E L, Meier P. Nonparametric Estimation from Incomplete Observations. J Am Stat Assoc. 1958;
18. Wild A T, Herman J M, Dholakia A S, Moningi S, Lu Y, Rosati L M, et al. Lymphocyte-Sparing Effect of Stereotactic Body Radiation Therapy in Patients with Unresectable Pancreatic Cancer. Int J Radiat Oncol Biol Phys. 2016; 94(3):571-9.
19. de Visser, K. E., Eichten, A., and Coussens, L. M. (2006). Paradoxical roles of the immune system during cancer development. Nat. Rev. Cancer 6, 24-37.
20. Pages, F., Galon, J., Dieu-Nosjean, M. C., Tartour, E., Sautes-Fridman, C., and Fridman, W. H. (2010). Immune infiltration in human tumors: a prognostic factor that should not be ignored. Oncogene 29, 1093-1102.
21. Oncology Meets Immunology: The Cancer-Immunity Cycle Daniel S. Chen and Ira Mellman Immunity 39, Jul. 25, 2013. http://dx.doi.org/10.1016/j.immuni.2013.07.12
22. Elements of cancer immunity and the cancer-immune set point Daniel S. Chen1& Ira Mellman1 2017|VOL 541|NATURE|321 doi:10.1038/nature21349
23. Zhao X, Yan X, Ju Y F, Yu H M, Jiao S C. [Expression and clinical significance of CD3, CD4 and COX-2 in non-small cell lung cancer]. Xi Bao Yu Fen Zi Mian Yi Xue Za Zhi. 2012; 28(4):407-9.
24. Pittet M J. Behavior of immune players in the tumor microenvironment. Vol. 21, Current Opinion in Oncology. 2009. P. 53-9.
25. Greenhough A, Smartt H J M, Moore A E, Roberts H R, Williams A C, Paraskeva C, et al. The COX-2/PGE2 pathway: Key roles in the hallmarks of cancer and adaptation to the tumour microenvironment. Carcinogenesis. 2009; 30(3):377-86.
26. Yu Q M, Yu C D, Ling Z Q. Elevated circulating CD19+ lymphocytes predict survival advantage in patients with gastric cancer. Asian Pac J Cancer Prev. 2012; 13(5):2219-24.
27. Kim H-J, Cantor H. CD4 T-cell Subsets and Tumor Immunity: The Helpful and the Not-so-Helpful. Cancer Immunol Res. 2014; 2(2):91-8.
28. Protti M P, Monte L D, Lullo G D. Tumor antigen-specific CD4+ T-cells in cancer immunity: From antigen identification to tumor prognosis and development of therapeutic strategies. Vol. 83, Tissue Antigens. 2014. P. 237-46.
29. Zhang L, Romero P. Metabolic Control of CD8+ T-cell Fate Decisions and Antitumor Immunity. Trends Mol Med. 2017;
30. Scheuermann R H, Racila E. CD19 antigen in leukemia and lymphoma diagnosis and immunotherapy. Leuk Lymphoma. 1995; 18(5-6):385-97.
31. Wagner J A, Rosario M, Romee R, Berrien-Elliott M M, Schneider S E, Leong J W, et al. CD56brightNK cells exhibit potent antitumor responses following IL-15 priming. J Clin Invest. 2017; 127(11).
32. Poli A, Michel T, Thérésine M, Andrès E, Hentges F, Zimmer J. CD56bright natural killer (NK) cells: An important NK cell subset. Vol. 126, Immunology. 2009. P. 458-65.
33. Ohba, K., Omagari, K., Nakamura, T., Ikuno, N., Saeki, S., Matsuo, I., et al. (1998). Abscopal regression of hepatocellular carcinoma after radiotherapy for bone metastasis. Gut 43, 575-577.
34. Nesslinger, N. J., Sahota, R. A., Stone, B., Johnson, K., Chima, N., King, C., et al. (2007). Standard treatments induce antigen-specific immune responses in prostate cancer. Clin. Cancer Res. 13, 1493-1502.
35. Kusmartsev, S., and Gabrilovich, D. I. (2002). Immature myeloid cells and cancer-associated immune suppression. Cancer Immunol. Immunother. 51, 293-298.
36. Chakravarty, P. K., Alfieri, A., Thomas, E. K., Beri, V., Tanaka, K. E., Vikram, B., et al. (1999). Flt3-ligand administration after radiation therapy prolongs survival in a murine model of metastatic lung cancer. Cancer Res. 59, 6028-6032.
37. Melero, I., Vile, R. G., and Colombo, M. P. (2000). Feeding dendritic cells with tumor antigens: self-service buffet or a la carte? Gene Ther. 7, 1167-1170.
38. Basu, S., Binder, R. J., Suto, R., Anderson, K. M., and Srivastava, P. K. (2000). Necrotic but not apoptotic cell death releases heat shock proteins, which deliver a partial maturation signal to dendritic cells and activate the NF-kappa B pathway. Int. Immunol. 12, 1539-1546.
39. Gattinoni, L., Finkelstein, S. E., Klebanoff, C. A., Antony, P. A., Palmer, D. C., Spiess, P. J., et al. (2005). Removal of homeostatic cytokine sinks by lymphodepletion enhances the efficacy of adoptively transferred tumor-specific CD8+ T-cells. J. Exp. Med. 202, 907-912.
40. Ganss, R., Ryschich, E., Klar, E., Arnold, B., and Hammerling, G. J. (2002). Combination of T-cell therapy and trigger of inflammation induces remodeling of the vasculature and tumor eradication. Cancer Res. 62, 1462-1470.
41. Reits, E. A., Hodge, J. W., Herberts, C. A., Groothuis, T. A., Chakraborty, M., Wansley, E. K., et al. (2006). Radiation modulates the peptide repertoire, enhances MHC class I expression, and induces successful antitumor immunotherapy. J. Exp. Med. 203, 1259-1271.
42. Apetoh, L., Ghiringhelli, F., Tesniere, A., Obeid, M., Ortiz, C., Criollo, A., et al. (2007). Tolllike receptor 4-dependent contribution of the immune system to anticancer chemotherapy and radiotherapy. Nat. Med. 13, 1050-1059.
43. Liao, Y. P., Wang, C. C., Schaue, D., Iwamoto, K. S., and McBride, W. H. (2009). Local irradiation of murine melanoma affects the development of tumour-specific immunity. Immunology 128(Suppl.), e797-e804.
44. Merrick, A., Errington, F., Milward, K., O'Donnell, D., Harrington, K., Bateman, A., et al. (2005). Immunosuppressive effects of radiation on human dendritic cells: reduced IL-12 production on activation and impairment of I T-cell priming. Br. J. Cancer 92, 1450-1458.
45. Chakraborty, M., Abrams, S. I., Coleman, C. N., Camphausen, K., Schlom, J., and Hodge, J. W. (2004). External beam radiation of tumors alters phenotype of tumor cells to render them susceptible to vaccine-mediated T-cell killing. Cancer Res. 64, 4328-4337.
46. Lugade, A. A., Moran, J. P., Gerber, S. A., Rose, R. C., Frelinger, J. G., and Lord, E. M. (2005). Local radiation therapy of B16 melanoma tumors increases the generation of tumor antigen specific effector cells that traffic to the tumor. J. Immunol. 174, 7516-7523.
47. Shiraishi, K., Ishiwata, Y., Nakagawa, K., Yokochi, S., Taruki, C., Akuta, T., et al. (2008). Enhancement of antitumor radiation efficacy and consistent induction of the abscopal effect in mice by ECI301, an active variant of macrophage inflammatory protein-1alpha. Clin. Cancer Res. 14, 1159-1166.
48. Cancer Immunology at the Crossroads: Complementary Therapeutic Modalities, Harnessing the Potential of Radiation-Induced Immune Modulation for Cancer Therapy Mansoor M. Ahmed, James W. Hodge, Chandan Guha, Eric J. Bernhard, Bhadrasain Vikram, and C. Norman Coleman, Cancer Immunol Res; 1(5); 280-4. 2013
49. Robbins, P. F., El-Gamil, M., Li, Y. F., Kawakami, Y., Loftus, D., Appella, E., et al. (1996). A mutated beta-catenin gene encodes a melanoma-specific antigen recognized by tumor infiltrating lymphocytes. J. Exp. Med. 183, 1185-1192.

### ADDITIONAL REFERENCES

The devices, systems, apparatuses, modules, compositions, materials, computer program products, non-transitory computer readable medium, and methods of various embodiments of the invention disclosed herein may utilize aspects (such as devices, apparatuses, modules, systems, compositions, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).
A. International Patent Application Publication No. WO 2021/051089 A1, Timmerman et al., “Personalized Ultra-Fractionated Stereotactic Adaptive Radiotherapy,” Mar. 18, 2021.
B. U.S. Patent Application Publication No. US 2019/0022411 A1, Parry et al., “Methods of Use of Ultra-High Dose Rate Radiation and Therapeutic Agent,”, Jan. 24, 2019.
C. U.S. Patent Application Publication No. US 2016/0129282 A1, Yin et al., “Systems and Methods for Specifying Treatment Criteria and Treatment Parameters for Patient Specific Radiation Therapy Planning”, May 12, 2016.
D. U.S. Pat. No. 10,549,116 B2, Sheng et al., “Radiotherapy Utilizing the Entire 4PI Solid Angle”, Feb. 4, 2020.
E. U.S. Pat. No. 6,366,798 B2, Green, “Radiotherapy Machine Including Magnetic Resonance Imaging System”, Apr. 2, 2002.
F. 50. U.S. Utility patent application Ser. No. 17/237,958, entitled “System, Method and Computer Readable Medium to Estimate the Post-Treatment Blood Cell Sub Type Count in Patients Treated via Radiation Therapy”, filed Apr. 22, 2021; Publication No. US 2021-0335496 A1, Oct. 28, 2021.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims) including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particular interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A computer method for determining a radiation therapy (RT) treatment plan for a subject, said method comprising:
a) determining, using subject data for the subject and a radiation therapy (RT) treatment plan simulation model, an internal target volume (ITV) mean fraction dosage within a first specified amount of radiation dose for a specified number of radiation therapy (RT) fractions;
b) determining, using subject data and the RT treatment plan simulation model, a total dose and/or an equivalent fractional dose to the heart that is less than a second specified amount of radiation;
c) determining, using the subject data and the RT treatment plan simulation model, a total dose and/or an equivalent fractional dose to one or more specified blood-rich organs that is less than a third specified amount of radiation;
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation;
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘f’; and
h) outputting said RT treatment plan for use in concurrently or subsequently treating the subject.

2. The method of claim 1, further comprising outputting said RT plan for use in concurrently or subsequently treating the subject with one or more of the following:
immunotherapy, surgery, or chemotherapy.

3. The method of claim 1, wherein said radiation therapy (RT) treatment plan is configured for use in concurrently or subsequently treating the subject with RT fractionation.

4. The method of claim 3, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

5. The method of claim 1, wherein said outputting the RT treatment plan comprises transmitting the RT treatment plan to any one or more of the following:
local memory;
remote memory; or
a display or a graphical user interface.

6. The method of claim 1, wherein said processing is accomplished by a computer processor or at least one computer.

7. The method of claim 1, wherein said method further comprises:
communicating with a server coupled to a network;
coupling a user interface to said network; and
coupling an application to said server and/or said user interface.

8. The method of claim 1, wherein said subject data comprises any one or more of the following:
anatomic imaging CT, MRI, ultrasound, or planar imaging,
functional imaging,
molecular imaging and optical imaging,
nuclear imaging,
pre-treatment immune cell levels,
structure set, and
Immune cells.

9. The method of claim 8, wherein said structure set comprises any one or more of the following:
gross target volume (GTV);
clinical Target Volume (CTV);
internal Target Volume (ITV);
planning Target Volume (PTV);
location of the tumor;
blood rich organs;
lymph rich organs;
primary lymphoid organs
secondary lymphoid organs; or
tumor draining lymph node.

10. The method of claim 8, wherein said immune cells comprise any one or more of the following
Lymphocytes;
CD4+ (aka Helper T-Cells);
CD8+ (aka Cytotoxic T-Cells, or Killer T-Cells);
Tregs (aka Regulatory T-Cells);
CD45+;
CD3+;
CD19+;
CD56+;
Naïve T and B cells;
Activated T and B cells;
Memory T and B cells;
Cytotoxic T cells;
B cells;
Natural Killer cells;
Dendritic cells;
Neutrophils;
Macrophages;
Monocytes;
mast cells; and
Eosinophil.

11. The method of claim 1, wherein said subject data comprises any one or more of the following patient factors:
subject age;
blood cell sub-type distribution;
pre-treatment rate of regeneration;
pre-treatment rate of redistribution;
type of radiation; or
molecular imaging.

12. The method of claim 1, wherein:
said keeping an internal target volume (ITV) mean fraction dosage within said specified amount of radiation is within the range of about 14Gy to about 17Gy for said specified number of RT fractions.

13. The method of claim 1, wherein:
said keeping the total dose to heart to less than said specified amount of radiation is about 5Gy.

14. The method of claim 1, wherein:
said keeping the equivalent fractional dose to the heart to less than said specified amount of radiation is about 5Gy/N where N is number of fractions.

15. The method of claim 1, wherein:
said specified blood-rich organs comprise one or more of the following: aorta, vena cava, pulmonary artery, liver, hepatic artery, and brain.

16. The method of claim 1, wherein:
said keeping the total dose to specified blood-rich organs to less than said specified amount of radiation is about 8Gy.

17. The method of claim 1, wherein:
said keeping the equivalent fractional dose to specified blood-rich organs to less than said specified amount of radiation is 8Gy/N where N is number of fractions.

18. The method of claim 1, wherein:
said specified bone-marrow-rich organs comprises any one or more of the following: spine, the pelvic bone, and the thigh bone.

19. The method of claim 1, wherein:
said keeping the total dose to specified bone-marrow-rich organs to less than said specified amount of radiation is about 2Gy.

20. The method of claim 1, wherein:
said keeping the equivalent fractional dose to specified bone-marrow-rich organs to less than said specified amount of radiation is about 40cGy.

21. The method of claim 1, wherein:
said secondary lymphoid organs comprise any one more of the following: lymph nodes, spleen, tonsils, Peyer's Patches, adenoids, nasal associated lymphoid tissues, and mucosal tissues.

22. The method of claim 1, wherein:
said keeping the total dose to specified secondary lymphoid organs to less than said specified amount of radiation is about 2Gy.

23. The method of claim 1, wherein:
said keeping the equivalent fractional dose to specified secondary lymphoid organs to less than said specified amount of radiation is about 40cGy.

24. The method of claim 1, wherein:
said keeping the total dose to tumor draining lymph node to less than said specified amount of radiation is about 2Gy.

25. The method of claim 1, wherein:
said keeping the equivalent fractional dose to tumor draining lymph node to less than said specified amount of radiation is about 40cGy.

26. The method of claim 1, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

27. The method of claim 1, wherein delivery of said radiation therapy (RT) treatment plan comprises any one or more of the following:
three-dimensional conformal radiation therapy (3DCRT) fractions;
intensity-modulated radiation therapy (IMRT) fractions;
volumetric modulated arc radiation therapy (VMAT) fractions; and
Rapid Arc fractions.

28. The method of claim 1, wherein said radiation therapy (RT) plan comprises molecular imaging.

29. The method of claim 1, wherein said subject patient data comprises any one or more of the following patient/tumor microenvironment factors that could determine the required mean fraction dosage to the internal target volume (ITV) which could be extracted from personalized medicine via artificial intelligence or machine learning:
subject age;
pre-Tx blood cell sub-type distribution;
amount of circulating tumor DNA (ct-DNA);
tumor PD-1, and PD-L1 expression;
tumor burden;
tumor type and stage;
type of radiation;
cytokines and cell surface markers in the tumor microenvironment;
proteomics;
radiomics; or
genomics.

30. A system for use in determining a radiation therapy (RT) treatment plan for a subject, said system comprising:
a computer processor;
a memory configured to store instructions that are executable by said computer processor, wherein said computer processor is configured to execute the instructions for:
a) determining, using subject data for the subject and a radiation therapy (RT) treatment plan simulation model, an internal target volume (ITV) mean fraction dosage within a specified amount of radiation dose for a first specified number of radiation therapy RT fractions;
b) determining, using the subject data and the RT treatment plan simulation model, a total dose and/or an equivalent fractional dose to the heart that is less than a second specified amount of radiation
c) determining, using the subject data and the RT treatment plan simulation model, a total dose and/or an equivalent fractional dose to one or more specified blood-rich organs that is less than a third specified amount of radiation
d) determining, using the subject data and the RT treatment plan simulation model, a total dose and/or an equivalent fractional dose to one or more specified bone-marrow-rich organs that is less than a fourth specified amount of radiation;
e) determining, using the subject data and the RT treatment plan simulation model, a total dose and/or an equivalent fractional dose to one or more specified secondary lymphoid organs that is less than a fifth specified amount of radiation
f) determining, using the subject data and the RT treatment plan simulation model, a total dose and/or an equivalent fractional dose to a tumor draining lymph node that is less than a sixth specified amount of radiation;
g) determining the radiation therapy (RT) treatment plan for the subject using results from the processing steps ‘a’ through ‘; and
h) outputting said RT treatment plan for use in concurrently or subsequently treating the subject.

31. The system of claim 30, wherein said system further comprises outputting said RT treatment plan for use in concurrently or subsequently treating the subject with one or more of the following:
immunotherapy, surgery, or chemotherapy.

32. The system of claim 33, wherein said radiation therapy (RT) treatment plan is configured for use in concurrently or subsequently treating the subject with RT fractionation.

33. The system of claim 32, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

34. The system of claim 30, wherein said outputting the RT treatment plan comprises transmitting the RT treatment plan to any one or more of the following:
local memory;
remote memory; or
a display or a graphical user interface.

35. The system of claim 30, wherein said processing is accomplished by a computer processor or at least one computer.

36. The system of claim 30, wherein said system further comprises:
communicating with a server coupled to a network;
coupling a user interface to said network; and
coupling an application to said server and/or said user interface.

37. The system of claim 30, wherein said memory comprises a main memory and a static memory.

38. The system of claim 30, wherein said memory comprises one or more of any one of the following:
electrically programmable read-only memory;
electrically erasable programmable read-only memory;
flash memory drive;
magnetic disk;
internal hard disk;
external hard disk;
solid-state drive;
removable disk;
magneto-optical disk;
CD-ROM disk; or
DVD-ROM disk.

39. The system of claim 30, wherein said subject data comprises any one or more of the following:
a) anatomic imaging CT, MRI, ultrasound, or planar imaging,
f) functional imaging,
g) molecular imaging and optical imaging,
h) nuclear imaging,
i) pre-treatment immune cell levels,
f) structure set, and
g) Immune cells.

40. The system of claim 39, wherein said structure set comprises any one or more of the following:
gross target volume (GTV);
clinical Target Volume (CTV);
internal Target Volume (ITV);
planning Target Volume (PTV);
location of the tumor;
blood rich organs;
lymph rich organs;
primary lymphoid organs;
secondary lymphoid organs or
tumor draining lymph node.

41. The system of claim 39, wherein said immune cells comprise any one or more of the following:
Lymphocytes;
CD4+ (aka Helper T-Cells);
CD8+ (aka Cytotoxic T-Cells, or Killer T-Cells);
Tregs (aka Regulatory T-Cells);
CD45+;
CD3+;
CD19+;
CD56+;
Naïve T and B cells;
Activated T and B cells;
Memory T and B cells;
Cytotoxic T cells;
B cells;
Natural Killer cells;
Dendritic cells;
Neutrophils;
Macrophages;
Monocytes;
mast cells; and
Eosinophil.

42. The system of claim 30, wherein said subject data comprises any one or more of the following patient factors:
subject age;
blood cell sub-type distribution;
pre-treatment rate of regeneration;
pre-treatment rate of redistribution;
type of radiation; or
molecular imaging.

43. The system of claim 30, wherein:
said keeping an internal target volume (ITV) mean fraction dosage within said specified amount of radiation is within the range of about 14Gy to about 17Gy for said specified number of RT fractions.

44. The system of claim 30, wherein:
said keeping the total dose to heart to less than said specified amount of radiation is about 5Gy.

45. The system of claim 30, wherein:
said keeping the equivalent fractional dose to the heart to less than said specified amount of radiation is about 5Gy/N where N is number of fractions.

46. The system of claim 30, wherein:
said specified blood-rich organs comprise one or more of the following: aorta, vena cava, pulmonary artery, liver, hepatic artery, and brain.

47. The system of claim 30, wherein:
said keeping the total dose to specified blood-rich organs to less than said specified amount of radiation is about 8Gy.

48. The system of claim 30, wherein:
said keeping the equivalent fractional dose to specified blood-rich organs to less than said specified amount of radiation is 8Gy/N where N is number of fractions.

49. The system of claim 30, wherein:
said specified bone-marrow-rich organs comprises any one or more of the following: spine, the pelvic bone, and the thigh bone.

50. The system of claim 30, wherein:
said keeping the total dose to specified bone-marrow-rich organs to less than said specified amount of radiation is about 2Gy.

51. The system of claim 30, wherein:
said keeping the equivalent fractional dose to specified bone-marrow-rich organs to less than said specified amount of radiation is about 40cGy.

52. The system of claim 30, wherein:
said secondary lymphoid organs comprise any one more of the following: lymph nodes, spleen, tonsils, Peyer's Patches, adenoids, nasal associated lymphoid tissues, and mucosal tissues.

53. The system of claim 30, wherein:
said keeping the total dose to specified secondary lymphoid organs to less than said specified amount of radiation is about 2Gy.

54. The system of claim 30, wherein:
said keeping the equivalent fractional dose to specified secondary lymphoid organs to less than said specified amount of radiation is about 40cGy.

55. The system of claim 30, wherein:
said keeping the total dose to tumor draining lymph node to less than said specified amount of radiation is about 2Gy.

56. The system of claim 30, wherein:
said keeping the equivalent fractional dose to tumor draining lymph node to less than said specified amount of radiation is about 40cGy.

57. The system of claim 30, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

58. The system of claim 30, wherein delivery of said radiation therapy (RT) treatment plan comprises any one or more of the following:
e) three-dimensional conformal radiation therapy (3DCRT) fractions;
f) intensity-modulated radiation therapy (IMRT) fractions;
g) volumetric modulated arc radiation therapy (VMAT) fractions; and
h) Rapid Arc fractions.

59. The system of claim 30, wherein said radiation therapy (RT) plan comprises molecular imaging.

60. The system of claim 30, wherein said subject patient data comprises any one or more of the following patient/tumor microenvironment factors that could determine the required mean fraction dosage to the internal target volume (ITV) which could be extracted from personalized medicine via artificial intelligence or machine learning:
subject age;
pre-Tx blood cell sub-type distribution;
amount of circulating tumor DNA (ct-DNA);
tumor PD-1, and PD-L1 expression;
tumor burden;
tumor type and stage;
type of radiation;
cytokines and cell surface markers in the tumor microenvironment;
proteomics;
radiomics; or
genomics.

61. A non-transitory, computer readable storage medium having instructions stored thereon for use in determining a radiation therapy (RT) treatment plan for a subject, that, when executed by a computer processor, cause the computer processor to:
a) determine, using subject data for the subject and a radiation therapy (RT) treatment plan simulation model, an internal target volume (ITV) mean fraction dosage within a specified amount of radiation dose for a first specified number of radiation therapy (RT) fractions;
b) determine, using the subject data and the RT treatment plan simulation model, a total dose and/or an equivalent fractional dose to the heart that is less than a second specified amount of radiation
c) determine, using the subject data and the RT treatment plan simulation model, a total dose and/or an equivalent fractional dose to one or more specified blood-rich organs that is less than a third specified amount of radiation
d) determine, using the subject data and the RT treatment plan simulation model, a total dose and/or an equivalent fractional dose to one or more specified bone-marrow-rich organs that is less than a fourth specified amount of radiation
e) determine, using the subject data and the RT treatment plan simulation model, a total dose and/or an equivalent fractional dose to one or more specified secondary lymphoid organs that is less than a fifth specified amount of radiation
f) determine, using the subject data and the RT treatment plan simulation model, a total dose and/or an equivalent fractional dose to a tumor draining lymph node that is less than a sixth specified amount of radiation;
g) determine the radiation therapy (RT) treatment plan for the subject using results from the processing steps ‘a’ through ‘f’; and
h) output said RT treatment plan for use in concurrently or subsequently treating the subject.

62. The computer readable storage medium of claim 61, wherein, when executed by the computer processor, causes the computer processor to output said RT treatment plan for use in concurrently or subsequently treating the subject with one or more of the following:
immunotherapy, surgery, or chemotherapy.

63. The computer readable storage medium of claim 61, wherein, said radiation therapy (RT) treatment plan is configured for use in concurrently or subsequently treating the subject with RT fractionation.

64. The computer readable storage medium of claim 63, wherein said RT fractionation comprises any one or more of the following, in conjunction with image-guided radiation therapy (IGRT) to allow for precision tumor and organ at risk targeting:
stereotactic radiosurgery (SRS),
stereotactic body radiation therapy (SBRT),
stereotactic radiation therapy (SRT),
stereotactic ablative radiation therapy (SABR),
spatially fractionated radiation therapy (SFRT),
grid therapy,
SFRT-SBRT-PATHY—stereotactic body radiation therapy of partial tumor irradiation (SBRT-PATHY),
microbeam radiation therapy (MRT),
minibeam radiation therapy (MBRT),
4D-SFRT, or
ultra high radiation therapy—flash radiotherapy.

65. The computer readable storage medium of claim 61, wherein said outputting the RT treatment plan comprises transmitting the RT treatment plan to any one or more of the following:
local memory;
remote memory; or
a display or a graphical user interface.

66. The computer readable storage medium of claim 61, wherein said computer processor comprises at least one computer.

67. The computer readable storage medium of claim 61, wherein, when executed by the computer processor, causes the computer processor to communicate with:
a server coupled to a network;
a user interface to said network; and
an application coupled to said server and/or said user interface.

68. The computer readable storage medium of claim 61, wherein said subject data comprises any one or more of the following:
a) anatomic imaging CT, MRI, ultrasound, or planar imaging,
c) functional imaging,
d) molecular imaging and optical imaging,
e) nuclear imaging,
e) pre-treatment immune cell levels,
f) structure set, and
g) Immune cells.

69. The computer readable storage medium of claim 68, wherein said structure set comprises any one or more of the following:
gross target volume (GTV);
clinical Target Volume (CTV);
internal Target Volume (ITV);
planning Target Volume (PTV);
location of the tumor;
blood rich organs;
lymph rich organs;
primary lymphoid organs;
secondary lymphoid organs; or
tumor draining lymph node.

70. The computer readable storage medium of claim 68, wherein said immune cells comprise any one or more of the following:
Lymphocytes;
CD4+ (aka Helper T-Cells);
CD8+ (aka Cytotoxic T-Cells, or Killer T-Cells);
Tregs (aka Regulatory T-Cells);
CD45+;
CD3+;
CD19+;
CD56+;
Naïve T and B cells;
Activated T and B cells;
Memory T and B cells;
Cytotoxic T cells;
B cells;
Natural Killer cells;
Dendritic cells;
Neutrophils;
Macrophages;
Monocytes;
mast cells; and
Eosinophil.

71. The computer readable storage medium of claim 61, wherein said subject data comprises any one or more of the following patient factors:
subject age;
blood cell sub-type distribution;
pre-treatment rate of regeneration;
pre-treatment rate of redistribution;
type of radiation; or
molecular imaging.

72. The computer readable storage medium of claim 61, wherein:
said keeping an internal target volume (ITV) mean fraction dosage within said specified amount of radiation is within the range of about 14Gy to about 17Gy for said specified number of RT fractions.

73. The computer readable storage medium of claim 61, wherein:
said keeping the total dose to heart to less than said specified amount of radiation is about 5Gy.

74. The computer readable storage medium of claim 61, wherein:
said keeping the equivalent fractional dose to the heart to less than said specified amount of radiation is about 5Gy/N where N is number of fractions.

75. The computer readable storage medium of claim 61, wherein:
said specified blood-rich organs comprise one or more of the following: aorta, vena cava, pulmonary artery, liver, hepatic artery, and brain.

76. The computer readable storage medium of claim 61, wherein:
said keeping the total dose to specified blood-rich organs to less than said specified amount of radiation is about 8Gy.

77. The computer readable storage medium of claim 61, wherein:
said keeping the equivalent fractional dose to specified blood-rich organs to less than said specified amount of radiation is 8Gy/N where N is number of fractions.

78. The computer readable storage medium of claim 61, wherein:
said specified bone-marrow-rich organs comprises any one or more of the following: spine, the pelvic bone, and the thigh bone.

79. The computer readable storage medium of claim 61, wherein:
said keeping the total dose to specified bone-marrow-rich organs to less than said specified amount of radiation is about 2Gy.

80. The computer readable storage medium of claim 61, wherein:
said keeping the equivalent fractional dose to specified bone-marrow-rich organs to less than said specified amount of radiation is about 40cGy.

81. The computer readable storage medium of claim 61, wherein:
said secondary lymphoid organs comprise any one more of the following: lymph nodes, spleen, tonsils, Peyer's Patches, adenoids, nasal associated lymphoid tissues, and mucosal tissues.

82. The computer readable storage medium of claim 61, wherein:
said keeping the total dose to specified secondary lymphoid organs to less than said specified amount of radiation is about 2Gy.

83. The computer readable storage medium of claim 61, wherein:
said keeping the equivalent fractional dose to specified secondary lymphoid organs to less than said specified amount of radiation is about 40cGy.

84. The computer readable storage medium of claim 61, wherein:
said keeping the total dose to tumor draining lymph node to less than said specified amount of radiation is about 2Gy.

85. The computer readable storage medium of claim 61, wherein:
said keeping the equivalent fractional dose to tumor draining lymph node to less than said specified amount of radiation is about 40cGy.

86. The computer readable storage medium of claim 61, wherein delivery of said radiation therapy (RT) treatment plan comprises any one or more of the following:
external beam radiation therapy (EBRT),
photon therapy,
proton therapy,
particle beam therapy,
ion beam therapy, and
brachytherapy.

87. The computer readable storage medium of claim 61, wherein delivery of said radiation therapy (RT) treatment plan comprises any one or more of the following:
e) three-dimensional conformal radiation therapy (3DCRT) fractions;
f) intensity-modulated radiation therapy (IMRT) fractions;
g) volumetric modulated arc radiation therapy (VMAT) fractions; and
h) Rapid Arc fractions.

88. The computer readable storage medium of claim 61, wherein said radiation therapy (RT) plan comprises molecular imaging.

89. The computer readable storage medium of claim 61, wherein said subject patient data comprises any one or more of the following patient/tumor microenvironment factors that could determine the required mean fraction dosage to the internal target volume (ITV) which could be extracted from personalized medicine via artificial intelligence or machine learning:
subject age;
pre-Tx blood cell sub-type distribution;
amount of circulating tumor DNA (ct-DNA);
tumor PD-1, and PD-L1 expression;
tumor burden;
tumor type and stage;
type of radiation;
cytokines and cell surface markers in the tumor microenvironment;
proteomics;
radiomics; or
genomics.

90. A method for treating a subject with an optimizing radiation therapy (RT) treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
whereby the subject is treated with an optimized RT treatment.

91. The method according to claim 90, further comprising treating the subject with one or more additional therapies selected from the group consisting of immunotherapy, surgery, and chemotherapy.

92. The method according to claim 91, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

93. The method according to claim 90, further comprising treating the subject with RT fractionation.

94. The method accordingly to any one of claims 90-93, wherein:
(i) the internal target volume (ITV) mean fraction dosage is about 14 to about 17 Gy per fraction; and/or
(ii) the total dose to the subject's heart, aorta, vena cava, and pulmonary artery is less than about 8 Gy, optionally less than about 5 Gy; and/or
(iii) the total dose to the subject's thoracic spine is less than about 2 Gy; and/or
(iv) the total dose to the subject's lymph nodes and/or lymphatics is less than about 2 Gy; and/or
(v) the total dose to the subject's one or more tumor draining lymph nodes is less than about 2 Gy.

95. A method for reducing the risk of developing Treatment Related Lymphopenia (TRL) and/or radiation induced immune suppression (RIIS) in a subject undergoing a cancer and/or a tumor treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment reduces the risk of developing TRL and/or RIIS in the subject relative to a standard RT treatment.

96. The method of claim 95, wherein the subject has a cancer and/or a tumor selected from the group consisting of glioblastoma, advanced stage non-small cell lung cancer (NSCLC), pancreatic cancer, and squamous cell carcinoma of the head and neck.

97. The method of claim 94 or claim 95, wherein the optimized RT treatment increases survival of the subject relative to a standard RT treatment.

98. The method according to any one of claims 95-97, further comprising treating the subject with one or more additional therapies selected from the group consisting of immunotherapy, surgery, and chemotherapy.

99. The method according to claim 98, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

100. A method for inducing tumor reactive T cells in a subject undergoing an anti-tumor treatment, the method comprising applying an optimized radiation therapy (RT) treatment to the subject, wherein based on subject data, the optimized RT treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
wherein the optimized RT treatment induces tumor reactive T cells in the subject.

101. The method according to claim 100, further comprising treating the subject with one or more additional therapies selected from the group consisting of immunotherapy, surgery, and chemotherapy.

102. The method according to claim 101, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.

103. Use of an optimized radiation therapy (RT) treatment for treating a subject with a disease, disorder, or condition for which radiation therapy would be appropriate, and/or for reducing the risk of developing Treatment Related Lymphopenia (TRL) and/or radiation induced immune suppression (RIIS), and/or for inducing reactive T cells, wherein the optimized radiation therapy (RT) treatment comprises an amount of radiation that provides:
a) an internal target volume (ITV) mean fraction dosage that is less than a specified amount for a specified number of radiation therapy (RT) fractions;
b) a total dose to the subject's heart and/or an equivalent fractional dose to the subject's heart that is less than a specified amount for the specified number of RT fractions;
c) the total dose to specified blood-rich organs and/or an equivalent fractional dose to specified blood-rich organs that is less than a specified amount for the specified number of RT fractions;
d) a total dose to specified bone-marrow-rich organs and/or an equivalent fractional dose to specified bone-marrow-rich organs that is less than a specified amount for the specified number of RT fractions;
e) a total dose to specified secondary lymphoid organs and/or an equivalent fractional dose to specified secondary lymphoid organs that is less than a specified amount for the specified number of RT fractions; and
f) a total dose to one or more tumor draining lymph nodes and/or an equivalent fractional dose to one or more tumor draining lymph nodes that is less than a specified amount for the specified number of RT fractions,
and further wherein the optimized radiation therapy (RT) treatment treats the disease, disorder, or condition; reduces the risk of developing Treatment Related Lymphopenia (TRL) and/or radiation induced immune suppression (RIIS); and/or induces reactive T cells in the subject.

104. The use according to claim 103, wherein the subject has a cancer and/or a tumor selected from the group consisting of glioblastoma, advanced stage non-small cell lung cancer (NSCLC), pancreatic cancer, and squamous cell carcinoma of the head and neck.

105. The use according to claim 102 or claim 103, wherein the optimized RT treatment increases survival of the subject relative to a standard RT treatment.

106. The use according to any one of claims 103-105, further comprising treating the subject with one or more additional therapies selected from the group consisting of immunotherapy, surgery, and chemotherapy.

107. The use according to claim 106, wherein the one or more additional therapies comprises administering to the subject an Immune Checkpoint Blockade (ICB) therapy, optionally an ICB therapy comprising administering an anti-CTLA-4 antibody, an anti-PD1 antibody, an anti-PD-L1 antibody, or any combination thereof.
